(19) United States
(12) Patent Application Publication
(10) Pub. No.: US 2025/0340541 A1
(43) Pub. Date: Nov. 6, 2025

(54) METHODS OF PREPARING MODULATORS OF THR-BETA

(71) Applicant: Aligos Therapeutics, Inc., South San Francisco, CA (US)

(72) Inventors: Vladimir Serebryany, Redwood City, CA (US); Michael Hunter Welch, San Ramon, CA (US)

(73) Assignee: Aligos Therapeutics, Inc., South San Francisco, CA (US)

(21) Appl. No.: 19/182,810

(22) Filed: Apr. 18, 2025

Related U.S. Application Data
(60) Provisional application No. 63/642,525, filed on May 3, 2024.

(51) Int. Cl. C07D 403/12

(52) U.S. Cl. CPC C07D 403/12

(57) ABSTRACT

Disclosed herein are methods of preparing modulators of THR-β.

### CROSS-REFERENCE TO RELATED APPLICATIONS

This application claims the benefit of priority to U.S. Provisional Patent Application Ser. No. 63/642,525, filed on May 3, 2024, the entire disclosure of which is hereby incorporated by reference herein.

### FIELD OF THE DISCLOSURE

The present disclosure is in the field of pharmaceutical compounds and their preparation. In particular, the present disclosure is in the field of THR-0 modulators and their preparation.

### BACKGROUND OF THE DISCLOSURE

In parallel with the global increase in obesity, metabolic dysfunction-associated steatotic liver disease (MASLD, formerly known as nonalcoholic fatty liver disease (NAFLD); MASLD and NAFLD are used interchangeably) is becoming the leading cause of chronic liver disease and liver transplantation worldwide [1,2]. MASLD is believed to affect 30% of the adult population and 70-80% of individuals who are obese and diabetic. MASLD is defined as excess liver fat accumulation greater than 5% induced by causes other than alcohol intake. MASLD progresses to liver inflammation (metabolic-associated steatohepatitis (MASH), formerly known as nonalcoholic steatohepatitis, NASH; MASH and NASH are used interchangeably) and fibrosis in a variable proportion of individuals, ultimately leading to liver failure and hepatocellular carcinoma (HCC) in susceptible individuals [3].

In the United States alone, MASH is the third most common indication for liver transplantation and is on a trajectory to become the most common [4]. The most important medical need in patients with MASLD and MASH is an effective treatment to halt the progression and possibly reverse fibrosis, which is the main predictor of liver disease evolution [5,6].

Thyroid hormone (TH) is essential for normal development, growth and metabolism of all vertebrates. Its effects are mediated principally through triiodothyronine (T3), which acts as a ligand for the TH receptors (TRs, or THRs) β1, β2 and α1 [7]. In the absence of ligand, TR first binds as a heterodimer or homodimer on TH response elements (TRE) located in the promoter regions of target genes, where it interacts with corepressors. Upon ligand binding, the TR homodimers are dissociated in favor of heterodimer formation with the retinoid-X receptor (RXR), resulting in release of the corepressors and recruitment of coactivators. This new complex attracts a large number of proteins which engage the RNA polymerase II in the transcription of the targeted genes.

Two different genetic loci, denoted THRA and THRB, are responsible for encoding multiple interrelated TR isoforms that have distinct tissue distributions and biological functions. The two major isoforms with the broadest level of tissue expression are TRα1 and TRβ1 [8]. While TRα1 is expressed first during fetal development and is widely expressed in adult tissues, TRβ1 appears later in development and displays highest expression in the adult liver, kidney, and lung [9]. TRα1 is a key regulator of cardiac output, whereas TRβ1 helps in the control of metabolism in the liver. Importantly, the natural thyroid hormone T3 activates both TRα1 and TRβ1 without any significant selectivity.

Design of thyromimetic small molecule agents led to the identification of TR (or THR) agonists with varying levels of TRβ selectivity despite high structural similarity between the ligand-binding domains for TRβ and TRα. TRβ selectivity achieved by some of these compounds resulted in an improved therapeutic index for lipid lowering relative to cardiac effects such as heart rate, cardiac hypertrophy, and contractility [10-12].

TRα and TRβ agonists are also used in indications other than liver-related disorders, as has been known in the art. For example, TRβ selective agonists may be useful in the treatment of X-linked adrenoleukodystrophy [13, 14].

### SUMMARY

Provided herein, in one aspect, is a method to prepare compound A:

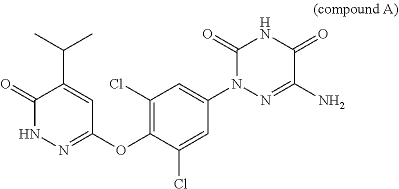 [SMILES: CC(C)c1cc(Oc2c(Cl)cc(-n3nc(N)c(=O)[nH]c3=O)cc2Cl)n[nH]c1=O]

wherein the method comprises converting compound 5:

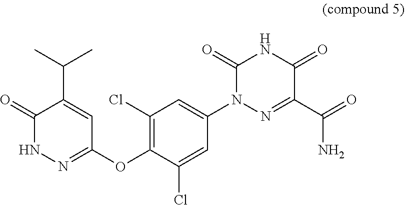 [SMILES: CC(C)c1cc(Oc2c(Cl)cc(-n3nc(C(N)=O)c(=O)[nH]c3=O)cc2Cl)n[nH]c1=O]

to compound A. In some embodiments, compound 5 is converted to compound A in a one-pot reaction. In some embodiments, compound 5 is converted to compound A in a single-step reaction. In some embodiments, compound 5 is converted to compound A under reaction conditions comprising contacting compound 5 with an oxidant and a base. In some embodiments, the oxidant comprises NaOCl. In some embodiments, the base comprises NaOH. In some embodiments, compound 5 is converted to compound A under inert gas.

In some embodiments, the method further comprises reacting compound 1:

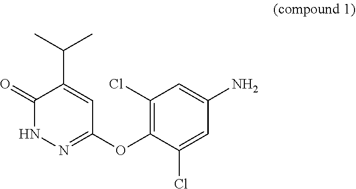 [SMILES: CC(C)c1cc(Oc2c(Cl)cc(N)cc2Cl)n[nH]c1=O]

with compound 2A:

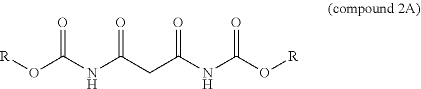

wherein R is C₁-C₃ alkyl or benzyl, under diazotization conditions to prepare compound 3A:

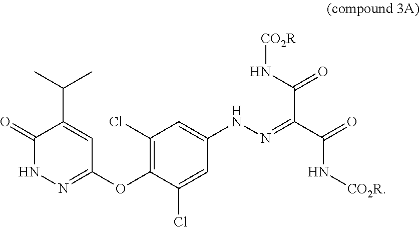

In some embodiments, the diazotization conditions comprise nitrite salt and one or more acids. In some embodiments, the diazotization conditions are under inert gas.

In some embodiments, the method further comprises cyclizing compound 3A to prepare compound 5 in a one-pot step comprising heating compound 3A under basic conditions. In some embodiments, the basic conditions are under inert gas. In some embodiments, the basic conditions comprise ammonia. In some embodiments, the basic conditions further comprise potassium carbonate. In some embodiments, the one-pot step comprises heating compound 3A at about 45° C. with ammonia and potassium carbonate in methanol.

In some embodiments, the method further comprises cyclizing compound 3A to prepare compound 6:

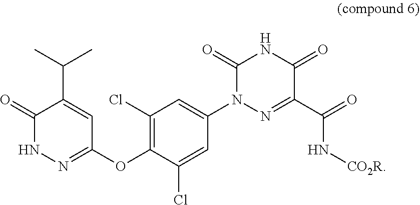

In some embodiments, the cyclizing is under inert gas. In some embodiments, the cyclizing is performed under refluxing buffered conditions. In some embodiments, the buffered conditions comprise aqueous acetic acid and acetate salt. In some embodiments, the buffered conditions comprise aqueous acetic acid and sodium acetate. In some embodiments, the method further comprises reacting compound 6 under basic conditions to prepare compound 5. In some embodiments, the basic conditions are under inert gas. In some embodiments, the basic conditions comprise ammonia. In some embodiments, the basic conditions further comprise potassium carbonate. In some embodiments, the basic conditions comprise methanol as solvent. In some embodiments, compound 6 is heated under basic conditions to prepare compound 5. In some embodiments, compound 6 is heated at about 50° C. to about 60° C. under basic conditions to prepare compound 5.

In some embodiments, the method further comprises reacting compound 1:

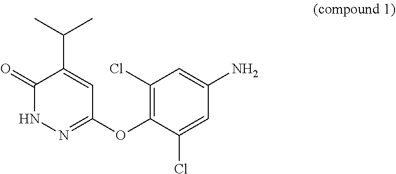 [SMILES: CC(C)c1cc(Oc2c(Cl)cc(N)cc2Cl)n[nH]c1=O]

with compound 2A:

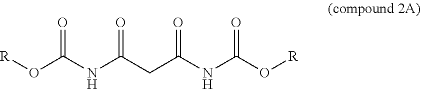

wherein R is C₁-C₃ alkyl or benzyl, under diazotization conditions followed by cyclizing under acidic conditions in a one-pot reaction to prepare compound 4:

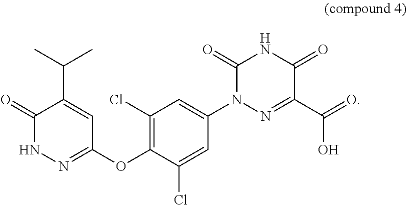 [SMILES: CC(C)c1cc(Oc2c(Cl)cc(-n3nc(C(=O)O)c(=O)[nH]c3=O)cc2Cl)n[nH]c1=O]

In some embodiments, the diazotization conditions comprise nitrite salt and one or more acids. In some embodiments, the diazotization conditions are under inert gas. In some embodiments, the acidic conditions comprise aqueous hydrogen chloride. In some embodiments, the cyclizing is performed at about 105° C. In some embodiments, the method further comprises reacting compound 4 under amidation conditions to prepare compound 5. In some embodiments, the amidation conditions comprise carbonyldiimidazole and NH₃ in dimethylformamide and water.

In some embodiments, the method further comprises reacting compound 1:

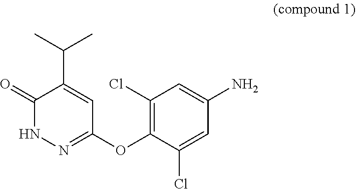 [SMILES: CC(C)c1cc(Oc2c(Cl)cc(N)cc2Cl)n[nH]c1=O]

with compound 2B:

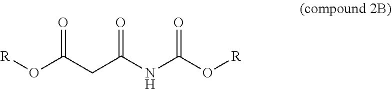

wherein R is C₁-C₃ alkyl or benzyl, under diazotization conditions to prepare compound 3B:

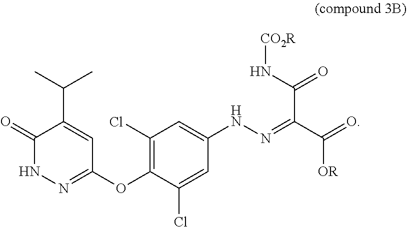

In some embodiments, the diazotization conditions comprise nitrite salt and one or more acids. In some embodiments, the diazotization conditions are under inert gas. In some embodiments, the method further comprises cyclizing compound 3B to prepare compound 7:

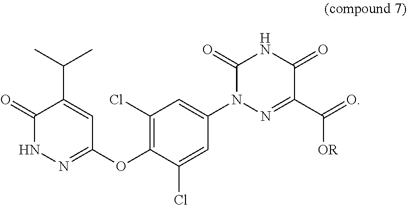

In some embodiments, the cyclizing is under inert gas. In some embodiments, the cyclizing is performed under refluxing buffered conditions. In some embodiments, the buffered conditions comprise aqueous acetic acid and acetate salt. In some embodiments, the buffered conditions comprise aqueous acetic acid and sodium acetate. In some embodiments, the method further comprises reacting compound 7 under basic conditions to prepare compound 5. In some embodiments, the basic conditions comprise ammonia. In some embodiments, the basic conditions comprise ammonia in methanol. In some embodiments, the basic conditions further comprise potassium carbonate. In some embodiments, compound 7 is reacted under basic conditions at room temperature to prepare compound 5.

In some embodiments, the method further comprises cyclizing compound 3C:

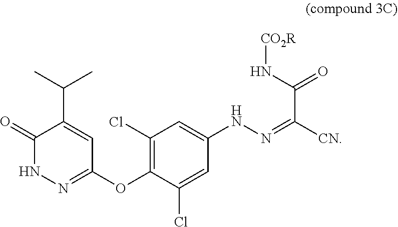

to prepare compound 5 in a one-pot step. In some embodiments, the one-pot step is under inert gas. In some embodiments, the one-pot step comprises refluxing buffered conditions, followed by heating under acidic conditions. In some embodiments, the buffered conditions comprise aqueous acetic acid and acetate salt. In some embodiments, the buffered conditions comprise aqueous acetic acid and sodium acetate. In some embodiments, the acidic conditions comprise trifluoracetic acid and sulfuric acid. In some embodiments, the heating under acidic conditions is conducted at about 80° C.

Provided herein, in another aspect, is a method to prepare compound A:

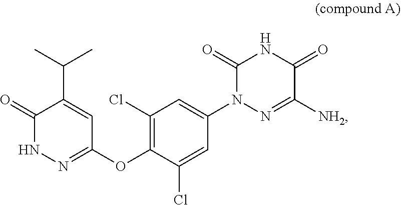 [SMILES: CC(C)c1cc(Oc2c(Cl)cc(-n3nc(N)c(=O)[nH]c3=O)cc2Cl)n[nH]c1=O]

wherein the method comprises:
   (1) adding bleach to a cooled aqueous solution of sodium hydroxide and compound 5:

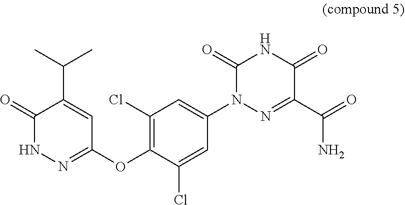 [SMILES: CC(C)c1cc(Oc2c(Cl)cc(-n3nc(C(N)=O)c(=O)[nH]c3=O)cc2Cl)n[nH]c1=O]

to form a reaction mixture;
   (2) stirring the reaction mixture while cooled with an ice bath;
   (3) stirring the reaction mixture at room temperature;
   (4) adjusting pH of the reaction mixture to pH of 8-9, with stirring, to form a slurry; and
   (5) isolating compound A from the slurry by filtration.
      In some embodiments, the cooled aqueous solution of sodium hydroxide and compound 5 is cooled to 0° C. to 7° C. In some embodiments, the cooled aqueous solution of sodium hydroxide and compound 5 is cooled to 4° C. to 5° C. In some embodiments, the reaction mixture is formed under inert gas. In some embodiments, the reaction mixture is stirred for about 1 hour while cooled with an ice bath. In some embodiments, the reaction mixture is stirred overnight (e.g., 8-15 hours) at room temperature. In some embodiments, the pH of the reaction mixture is adjusted to pH of 8-9 using acetic acid. In some embodiments, the method further comprises recrystallizing compound A under methanolic conditions. In some embodiments, the methanolic conditions comprise methanol, triethylamine, and acetic acid.

Provided herein, in another aspect, is a method to prepare compound A:

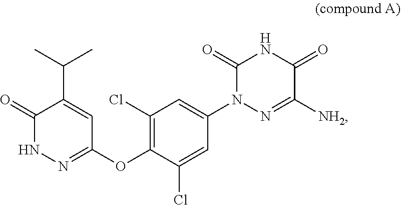 [SMILES: CC(C)c1cc(Oc2c(Cl)cc(-n3nc(N)c(=O)[nH]c3=O)cc2Cl)n[nH]c1=O]

wherein the method comprises:
   (1) adding acetic acid, water, and aqueous HCl to compound 1 and compound 2A-Et:

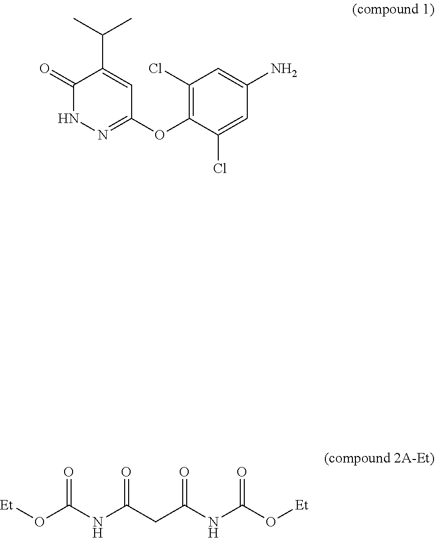 [SMILES: CC(C)c1cc(Oc2c(Cl)cc(N)cc2Cl)n[nH]c1=O ; CCOC(=O)NC(=O)CC(=O)NC(=O)OCC]

to form an initial reaction mixture;
   (2) cooling the initial reaction mixture with an ice bath and then adding an aqueous solution of sodium nitrite to the initial reaction mixture to form a second reaction mixture while maintaining the cooling by ice bath;
   (3) stirring the second reaction mixture while cooled with the ice bath;
   (4) adding sodium acetate to the second reaction mixture while cooled with the ice bath to form a third reaction mixture;
   (5) stirring the third reaction mixture while cooled with an ice bath;
   (6) stirring the third reaction mixture at room temperature;
   (7) isolating compound 3A-Et:

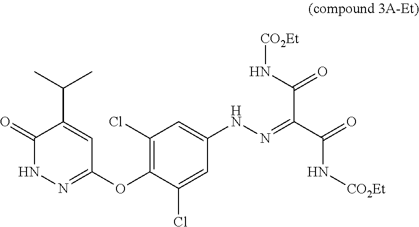 [SMILES: CCOC(=O)NC(=O)C(=NNc1cc(Cl)c(Oc2cc(C(C)C)c(=O)[nH]n2)c(Cl)c1)C(=O)NC(=O)OCC]

by filtration;
   (8) adding ammonia in methanol to compound 3A-Et and potassium carbonate to form a fourth reaction mixture;
   (9) stirring the fourth reaction mixture at about 45° C.;
   (10) adding water to the fourth reaction mixture at room temperature;
   (11) isolating compound 5:

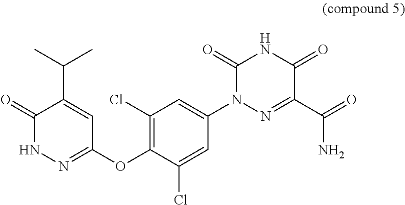 [SMILES: CC(C)c1cc(Oc2c(Cl)cc(-n3nc(C(N)=O)c(=O)[nH]c3=O)cc2Cl)n[nH]c1=O]

by filtration;
   (12) adding bleach to a cooled aqueous solution of sodium hydroxide and compound 5 to form a fifth reaction mixture;
   (13) stirring the fifth reaction mixture while cooled with an ice bath;
   (14) stirring the fifth reaction mixture at room temperature;
   (15) adjusting pH of the fifth reaction mixture to pH of 8-9, with stirring; and
   (16) isolating compound A by filtration.

In some embodiments, the initial reaction mixture is formed under inert gas. In some embodiments, the second reaction mixture is stirred for about 30 minutes while cooled with the ice bath. In some embodiments, the third reaction mixture is stirred for about 70 minutes while cooled with the ice bath. In some embodiments, the third reaction mixture is stirred overnight (e.g., 8-15 hours) at room temperature. In some embodiments, the fourth reaction mixture is formed under inert gas. In some embodiments, the fourth reaction mixture is stirred for about 2 hours at about 45° C. In some embodiments, the method further comprises recrystallizing compound 5. In some embodiments, compound 5 is recrystallized using DMSO, acetic acid, and water. In some embodiments, the cooled aqueous solution of sodium hydroxide and compound 5 is cooled to 0° C. to 7° C. In some embodiments, the cooled aqueous solution of sodium hydroxide and compound 5 is cooled to 4° C. to 5° C. In some embodiments, the fifth reaction mixture is formed under inert gas. In some embodiments, the fifth reaction mixture is stirred for about 1 hour while cooled with an ice bath. In some embodiments, the fifth reaction mixture is stirred overnight (e.g., 8-15 hours) at room temperature. In some embodiments, the pH of the fifth reaction mixture is adjusted to pH of 8-9 using acetic acid. In some embodiments, the method further comprises recrystallizing compound A under methanolic conditions. In some embodiments, the methanolic conditions comprise, consist essentially of, or consist of methanol, triethylamine, and acetic acid.

Provided herein, in another aspect, is a method to prepare compound A:

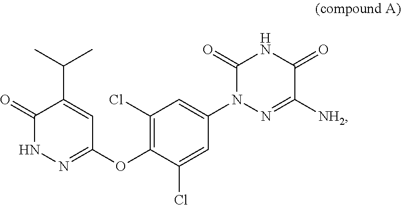 [SMILES: CC(C)c1cc(Oc2c(Cl)cc(-n3nc(N)c(=O)[nH]c3=O)cc2Cl)n[nH]c1=O]

wherein the method comprises:
   (1) adding bleach to a cooled aqueous solution of sodium hydroxide and compound 5:

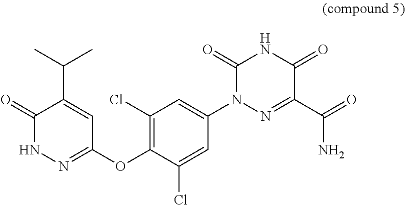 [SMILES: CC(C)c1cc(Oc2c(Cl)cc(-n3nc(C(N)=O)c(=O)[nH]c3=O)cc2Cl)n[nH]c1=O]

to form a reaction mixture;
   (2) stirring the reaction mixture while cooled with an ice bath;
   (3) stirring the reaction mixture at 35-45° C.;
   (4) adjusting pH of the reaction mixture to pH of 5-6, with stirring, to form a slurry; and
   (5) isolating compound A from the slurry by filtration.
      In some embodiments, the cooled aqueous solution of sodium hydroxide and compound 5 is cooled to 4° C. to 5° C. In some embodiments, the reaction mixture is formed under inert gas. In some embodiments, the reaction mixture is stirred for about 90 minutes while cooled with an ice bath. In some embodiments, the reaction mixture is stirred for about 6 hours at 35-45° C. In some embodiments, pH of the reaction mixture is adjusted to 5-6 using hydrochloric acid. In some embodiments, the method further comprises triturating compound A using acetic acid and a combination of THF and acetic acid.

Provided herein, in another aspect, is a method to prepare compound A:

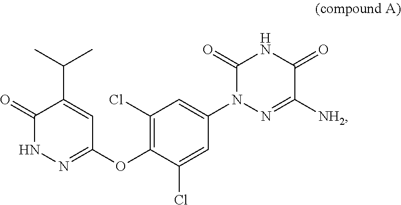 [SMILES: CC(C)c1cc(Oc2c(Cl)cc(-n3nc(N)c(=O)[nH]c3=O)cc2Cl)n[nH]c1=O]

wherein the method comprises:
(1) adding acetic acid, water, and aqueous HCl to compound 1 and compound 2A-Et:

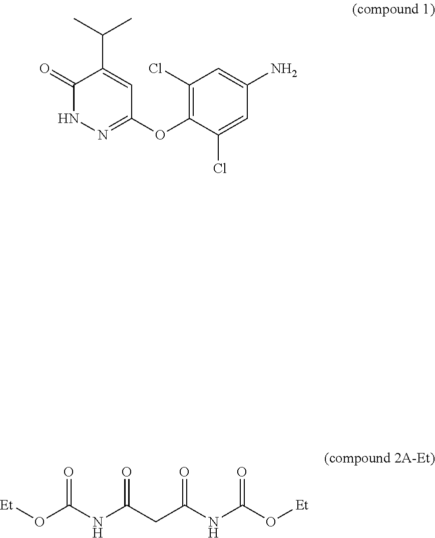 [SMILES: CC(C)c1cc(Oc2c(Cl)cc(N)cc2Cl)n[nH]c1=O ; CCOC(=O)NC(=O)CC(=O)NC(=O)OCC]

to form an initial reaction mixture;
   (2) adding an aqueous solution of sodium nitrite to the initial reaction mixture to form a second reaction mixture while maintaining the temperature at room temperature;
   (3) stirring the second reaction mixture at room temperature;
   (4) adding sodium acetate to the second reaction mixture while maintaining the temperature at no more than 30° C. to form a third reaction mixture;
   (5) stirring the third reaction mixture at room temperature;
   (6) isolating compound 3A-Et:

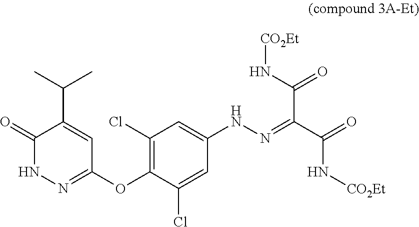 [SMILES: CCOC(=O)NC(=O)C(=NNc1cc(Cl)c(Oc2cc(C(C)C)c(=O)[nH]n2)c(Cl)c1)C(=O)NC(=O)OCC]

by filtration;
   (7) adding ammonia in methanol to compound 3A-Et and potassium carbonate to form a fourth reaction mixture;
   (8) stirring the fourth reaction mixture at about 45° C.;
   (9) adjusting the pH of the fourth reaction mixture to pH of 5-6 while maintaining the temperature at room temperature;
   (10) isolating compound 5:

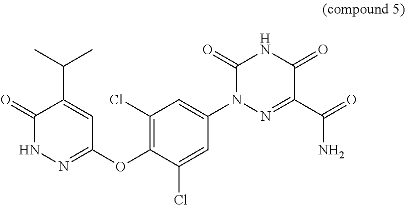 [SMILES: CC(C)c1cc(Oc2c(Cl)cc(-n3nc(C(N)=O)c(=O)[nH]c3=O)cc2Cl)n[nH]c1=O]

by filtration;
   (11) adding bleach to a cooled aqueous solution of sodium hydroxide and compound 5 to form a fifth reaction mixture;
   (12) stirring the fifth reaction mixture while cooled with an ice bath;
   (13) stirring the fifth reaction mixture at 35-45° C.;
   (14) adjusting pH of the fifth reaction mixture to pH of 5-6, with stirring, to form a slurry; and
   (15) isolating compound A from the slurry by filtration.
      In some embodiments, the initial reaction mixture is formed under inert gas. In some embodiments, the second reaction mixture is stirred for about 30 minutes at room temperature. In some embodiments, the third reaction mixture is stirred overnight at room temperature. In some embodiments, the fourth reaction mixture is formed under inert gas. In some embodiments, the fourth reaction mixture is stirred for at least 18 hours at about 45° C. In some embodiments, the method further comprises triturating compound 5. In some embodiments, compound 5 is triturated using tetrahydrofuran (THF). In some embodiments, the cooled aqueous solution of sodium hydroxide and compound 5 is cooled to 4° C. to 5° C. In some embodiments, the fifth reaction mixture is formed under inert gas. In some embodiments, the fifth reaction mixture is stirred for about 90 minutes while cooled with an ice bath. In some embodiments, the fifth reaction mixture is stirred for about 6 hours at 35-45° C. In some embodiments, the pH of the fifth reaction mixture is adjusted to pH of 5-6 using hydrochloric acid. In some embodiments, the method further comprises triturating compound A using acetic acid and a combination of THF and acetic acid.

Provided herein, in another aspect, is a compound having the structure of:

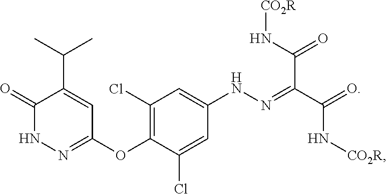

wherein R is C₁-C₃ alkyl or benzyl.

Provided herein, in another aspect, is a compound having the structure of:

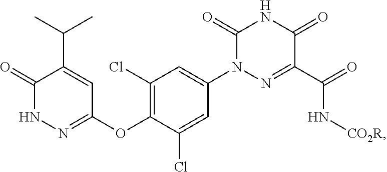

wherein R is C₁-C₃ alkyl or benzyl.

Provided herein, in another aspect, is a compound having the structure of:

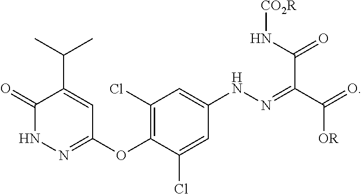

wherein R is C₁-C₃ alkyl or benzyl.

### DETAILED DESCRIPTION

### Definitions

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein and unless otherwise indicated, the term “single-step reaction” refers to the use of a single set of reagent(s) to effect a chemical transformation of a starting compound to a final product compound. In some embodiments, an intermediate compound is observed by chromatography of a sample from a reaction mixture undergoing a single-step reaction. In some embodiments, an intermediate compound is not observed by chromatography of a sample from a reaction mixture undergoing a single-step reaction.

As used herein and unless otherwise indicated, the term “one-pot reaction” refers to the use of more than one set of reaction conditions to effect multiple sequential chemical transformations of a starting compound to a final product compound within the same reaction vessel without isolation of any intermediate compound(s). A one-pot reaction may include 2, 3, 4, 5, 6, or 7 sequential chemical transformations, with each chemical transformation effected by separate reaction conditions. Each of the sets of reaction conditions may differ in the identity of the reagent(s), temperature, solvent, reaction time, or a combination of two or more thereof.

Unless otherwise indicated, the abbreviations “TR” and “THR” refer to thyroid hormone receptors.

As used herein, “pharmaceutically acceptable salt” refers to a salt of a compound that does not cause significant irritation to a patient to which it is administered and does not abrogate the biological activity and properties of the compound. Pharmaceutical salts can be obtained by reaction of a compound disclosed herein with an acid or base. Base-formed salts include, without limitation, ammonium salt (NH₄+); alkali metal, such as, without limitation, sodium or potassium, salts; alkaline earth, such as, without limitation, calcium or magnesium, salts; salts of organic bases such as, without limitation, dicyclohexylamine, N-methyl-D-glucamine, tris(hydroxymethyl)methylamine; and salts with the amino group of amino acids such as, without limitation, arginine and lysine. Useful acid-based salts include, without limitation, hydrochlorides, hydrobromides, sulfates, nitrates, phosphates, methane-sulfonates, ethanesulfonates, p-toluenesulfonates and salicylates.

Where the compounds disclosed herein have at least one chiral center, they may exist as a racemate or as individual enantiomers. It should be noted that all such isomers and mixtures thereof are included in the scope of the present disclosure. Thus, the illustration of a chiral center without a designation of R or S signifies that the scope of the disclosure includes the R isomer, the S isomer, the racemic mixture of the isomers, or mixtures where one isomer is present in greater abundance than the other.

Where the processes for the preparation of the compounds disclosed herein give rise to mixtures of stereoisomers, such isomers may be separated by conventional techniques such as preparative chiral chromatography. The compounds may be prepared in racemic form or individual enantiomers may be prepared by stereoselective synthesis or by resolution. The compounds may be resolved into their component enantiomers by standard techniques, such as the formation of diastereomeric pairs by salt formation with an optically active acid, such as (−)-di-p-toluoyl-d-tartaric acid and/or (+)-di-p-toluoyl-1-tartaric acid, followed by fractional crystallization and regeneration of the free base. The compounds may also be resolved by formation of diastereomeric esters or amides followed by chromatographic separation and removal of the chiral auxiliary.

It is understood that, in any compound of the presently disclosed compounds having one or more chiral centers, if an absolute stereochemistry is not expressly indicated, then each center may independently be R or S or a mixture thereof. In addition, it is understood that, in any compound of the presently disclosed compounds having one or more double bond(s) generating geometrical isomers that can be defined as E or Z each double bond may independently be E or Z, or a mixture thereof.

### Methods of Preparation

In one aspect, provided herein are methods to prepare compound A:

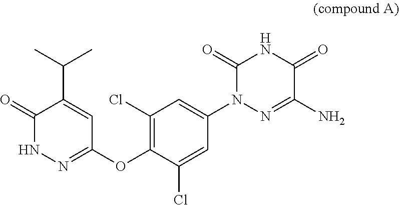 [SMILES: CC(C)c1cc(Oc2c(Cl)cc(-n3nc(N)c(=O)[nH]c3=O)cc2Cl)n[nH]c1=O]

or a tautomer thereof; or a pharmaceutically acceptable salt of the compound or the tautomer thereof. Compound A is also known as 6-amino-2-[3,5-dichloro-4-[(5-isopropyl-6-oxo-1H-pyridazin-3-yl)oxy]phenyl]-4H-1,2,4-triazine-3,5-dione.

In another aspect, provided herein is a method to prepare compound A, wherein the method comprises converting compound 5:

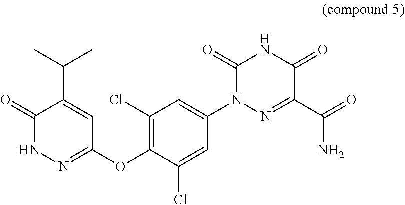 [SMILES: CC(C)c1cc(Oc2c(Cl)cc(-n3nc(C(N)=O)c(=O)[nH]c3=O)cc2Cl)n[nH]c1=O]

to compound A. In some embodiments, compound 5 is converted to compound A in a one-pot reaction. In some embodiments, compound 5 is converted to compound A in a single-step reaction.

In some embodiments, converting compound 5 to compound A is accomplished through a Hoffmann rearrangement. In some embodiments, compound 5 is converted to compound A under reaction conditions comprising, consisting essentially of, or consisting of contacting compound 5 with an oxidant and a base. Non-limiting examples of base include sodium hydroxide, lithium hydroxide, potassium hydroxide, and any combination of two or more thereof. Non-limiting examples of oxidant include NaOCl (e.g., bleach), bromine, or a combination thereof. In some embodiments, compound 5 is converted to compound A under reaction conditions comprising, consisting essentially of, or consisting of contacting compound 5 with N-chlorosuccinimide, N-bromosuccinimide, (diacetoxyiodo)benzene, or (bis(trifluoroacetoxy)iodo)-benzene. In some embodiments, converting compound 5 to compound A is conducted with stirring while ice-bath cooling, followed by stirring at room temperature. In some embodiments, converting compound 5 to compound A is conducted with stirring while ice-bath cooling, followed by stirring at 35-45° C. In some embodiments, compound 5 is converted to compound A under inert gas (e.g., argon or nitrogen).

In another aspect, provided herein is a method to prepare compound A:

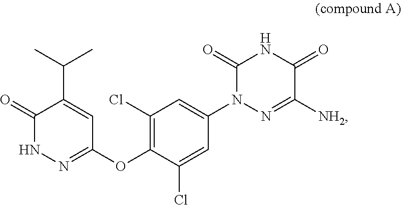 [SMILES: CC(C)c1cc(Oc2c(Cl)cc(-n3nc(N)c(=O)[nH]c3=O)cc2Cl)n[nH]c1=O]

wherein the method comprises:
   (1) adding bleach to a cooled aqueous solution of sodium hydroxide and compound 5:

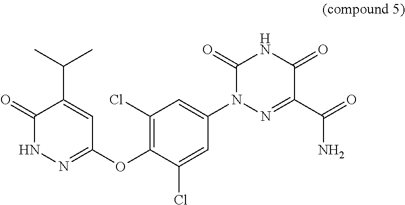 [SMILES: CC(C)c1cc(Oc2c(Cl)cc(-n3nc(C(N)=O)c(=O)[nH]c3=O)cc2Cl)n[nH]c1=O]

to form a reaction mixture;
   (2) stirring the reaction mixture while cooled with an ice bath;
   (3) stirring the reaction mixture at room temperature;
   (4) adjusting pH of the reaction mixture to pH of 8-9, with stirring, to form a slurry; and
   (5) isolating compound A from the slurry by filtration.
      In some embodiments, the cooled aqueous solution of sodium hydroxide and compound 5 is under inert gas (e.g., argon or nitrogen). In some embodiments, the cooled aqueous solution of sodium hydroxide and compound 5 is cooled to 0° C. to 7° C. In some embodiments, the cooled aqueous solution of sodium hydroxide and compound 5 is cooled to 4° C. to 5° C. In some embodiments, the reaction mixture is formed under inert gas (e.g., argon or nitrogen). In some embodiments, the reaction mixture is stirred for about 1 hour while cooled with an ice bath. In some embodiments, the reaction mixture is stirred overnight (e.g., 8-15 hours) at room temperature. In some embodiments, the pH of the reaction mixture is adjusted to pH of 8-9 using acetic acid. In some embodiments, the method further comprises recrystallizing compound A under methanolic conditions. In some embodiments, the methanolic conditions comprise, consist essentially of, or consist of methanol, triethylamine, and acetic acid.

In another aspect, provided herein is a method to prepare compound A:

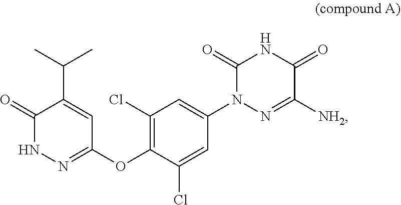 [SMILES: CC(C)c1cc(Oc2c(Cl)cc(-n3nc(N)c(=O)[nH]c3=O)cc2Cl)n[nH]c1=O]

wherein the method comprises:
   (1) adding bleach to a cooled aqueous solution of sodium hydroxide and compound 5:

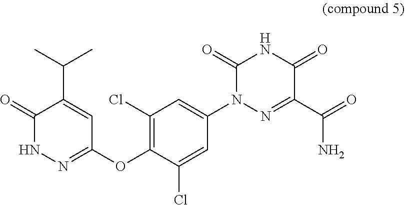 [SMILES: CC(C)c1cc(Oc2c(Cl)cc(-n3nc(C(N)=O)c(=O)[nH]c3=O)cc2Cl)n[nH]c1=O]

to form a reaction mixture;
   (2) stirring the reaction mixture while cooled with an ice bath;
   (3) stirring the reaction mixture at 35-45° C.;
   (4) adjusting pH of the reaction mixture to pH of 5-6, with stirring, to form a slurry; and
   (5) isolating compound A from the slurry by filtration.
      In some embodiments, the cooled aqueous solution of sodium hydroxide and compound 5 is under inert gas (e.g., argon or nitrogen). In some embodiments, the cooled aqueous solution of sodium hydroxide and compound 5 is cooled to 4° C. to 5° C. In some embodiments, the reaction mixture is formed under inert gas (e.g., argon or nitrogen). In some embodiments, the reaction mixture is stirred for about 90 min while cooled with an ice bath. In some embodiments, the reaction mixture is stirred for about 6 hours at 35-45° C. In some embodiments, pH of the reaction mixture is adjusted to 5-6 using hydrochloric acid. In some embodiments, the method further comprises triturating compound A using acetic acid and a combination of THF and acetic acid.

In some embodiments, compound A is prepared according to Scheme 1.

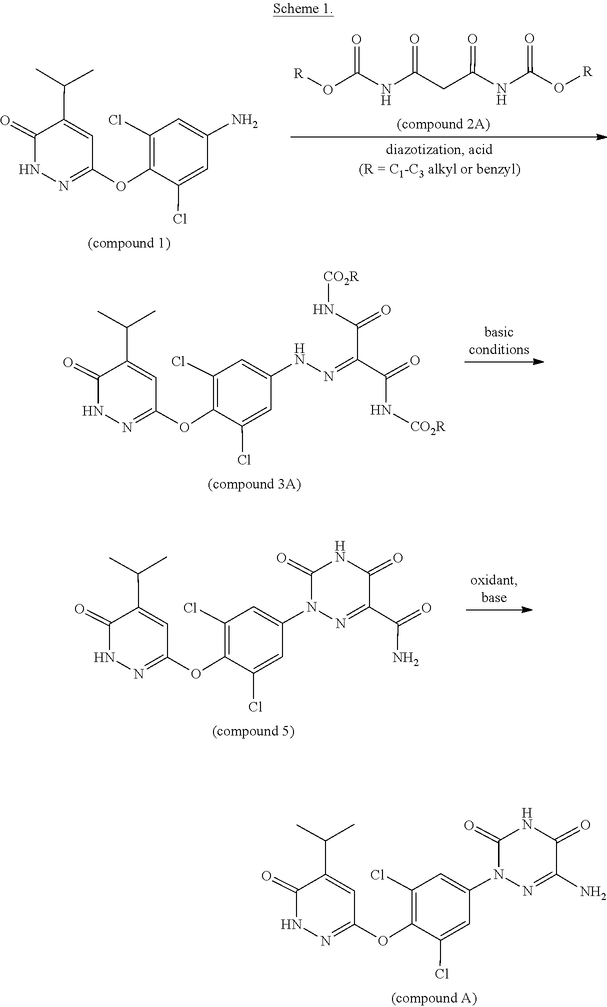

Accordingly, in some embodiments, the method to prepare compound A further comprises reacting compound 1:

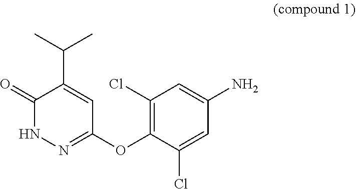 [SMILES: CC(C)c1cc(Oc2c(Cl)cc(N)cc2Cl)n[nH]c1=O]

with compound 2A:

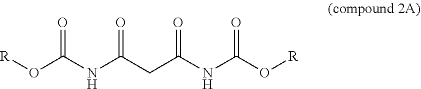

wherein R is C₁-C₃ alkyl or benzyl, under diazotization conditions to prepare compound 3A:

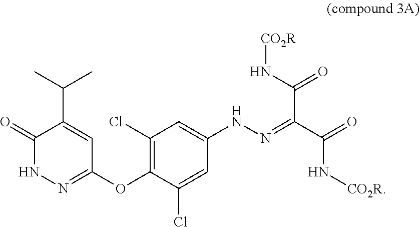

In some embodiments, R is C₁-C₃ alkyl. In some embodiments, R is methyl or ethyl. In some embodiments, R is ethyl. In some embodiments, R is methyl. In some embodiments, R is isopropyl. In some embodiments, R is benzyl.

In some embodiments, the diazotization conditions comprise nitrite salt and one or more acids. In some embodiments, the diazotization conditions comprise sodium nitrite and hydrochloric acid. In some embodiments, the diazotization conditions comprise an alkyl nitrite (e.g., isoamyl nitrite or tert-butyl nitrite) and one or more acids. In some embodiments, the diazotization conditions are under inert gas (e.g., argon or nitrogen). In some embodiments, compound 1 in aqueous acetic acid is reacted with compound 2A in the presence of sodium nitrite and hydrochloric acid prior to addition of sodium acetate to prepare compound 3A. In some embodiments, compound 1 in aqueous acetic acid is reacted with sodium nitrite and hydrochloric acid prior to reaction with compound 2A to prepare compound 3A.

In some embodiments, the method to prepare compound A further comprises cyclizing compound 3A to prepare compound 5 in a one-pot step comprising, consisting essentially of, or consisting of heating compound 3A under basic conditions. In some embodiments, wherein the basic conditions are under inert gas (e.g., argon or nitrogen). In some embodiments, the basic conditions comprise ammonia. In some embodiments, the basic conditions further comprise potassium carbonate, cesium carbonate, sodium methoxide, potassium methoxide, potassium tert-butoxide, or a combination of two or more thereof. In some embodiments, the basic conditions further comprise potassium carbonate. In some embodiments, the basic conditions comprise, consist essentially of, or consist of ammonia and potassium carbonate in methanol. In some embodiments, the basic conditions comprise, consist essentially of, or consist of ammonia in 1,4-dioxane or aqueous ammonia. In some embodiments, the one-pot step comprises heating compound 3A at about 45° C. with ammonia and potassium carbonate in methanol.

Accordingly, in another aspect, provided herein is a method to prepare compound A:

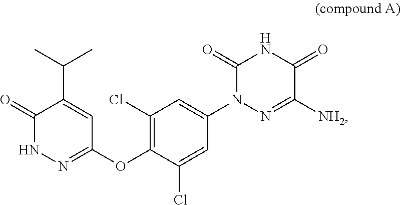 [SMILES: CC(C)c1cc(Oc2c(Cl)cc(-n3nc(N)c(=O)[nH]c3=O)cc2Cl)n[nH]c1=O]

wherein the method comprises:
   (1) adding acetic acid, water, and aqueous HCl to compound 1 and compound 2A-Et:

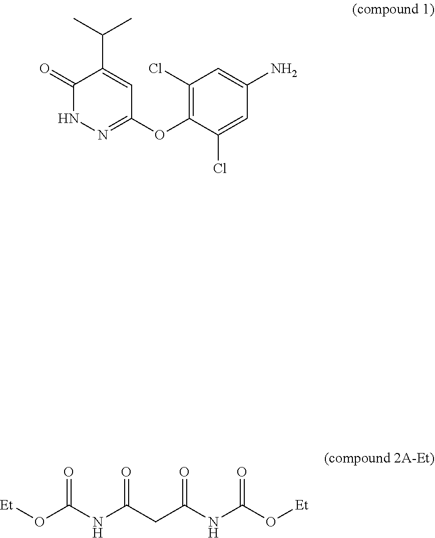 [SMILES: CC(C)c1cc(Oc2c(Cl)cc(N)cc2Cl)n[nH]c1=O ; CCOC(=O)NC(=O)CC(=O)NC(=O)OCC]

to form an initial reaction mixture;
   (2) cooling the initial reaction mixture with an ice bath and then adding an aqueous solution of sodium nitrite to the initial reaction mixture to form a second reaction mixture while maintaining the cooling by ice bath;
   (3) stirring the second reaction mixture while cooled with the ice bath;
   (4) adding sodium acetate to the second reaction mixture while cooled with the ice bath to form a third reaction mixture;
   (5) stirring the third reaction mixture while cooled with an ice bath;
   (6) stirring the third reaction mixture at room temperature;
   (7) isolating compound 3A-Et:

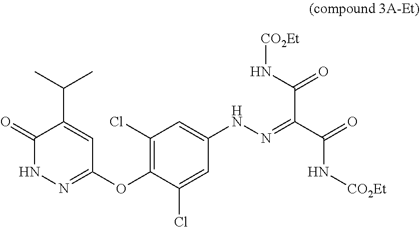 [SMILES: CCOC(=O)NC(=O)C(=NNc1cc(Cl)c(Oc2cc(C(C)C)c(=O)[nH]n2)c(Cl)c1)C(=O)NC(=O)OCC]

by filtration;
   (8) adding ammonia in methanol to compound 3A-Et and potassium carbonate to form a fourth reaction mixture;
   (9) stirring the fourth reaction mixture at about 45° C.;
   (10) adding water to the fourth reaction mixture at room temperature;
   (11) isolating compound 5:

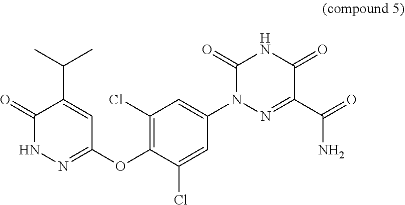 [SMILES: CC(C)c1cc(Oc2c(Cl)cc(-n3nc(C(N)=O)c(=O)[nH]c3=O)cc2Cl)n[nH]c1=O]

by filtration;
   (12) adding bleach to a cooled aqueous solution of sodium hydroxide and compound 5 to form a fifth reaction mixture;
   (13) stirring the fifth reaction mixture while cooled with an ice bath;
   (14) stirring the fifth reaction mixture at room temperature;
   (15) adjusting pH of the fifth reaction mixture to pH of 8-9, with stirring; and
   (16) isolating compound A by filtration.

In some embodiments, the initial reaction mixture is formed under inert gas (e.g., argon or nitrogen). In some embodiments, the second reaction mixture is stirred for about 30 minutes while cooled with the ice bath. In some embodiments, the third reaction mixture is stirred for about 70 minutes while cooled with the ice bath. In some embodiments, the third reaction mixture is stirred overnight (e.g., 8-15 hours) at room temperature. In some embodiments, the fourth reaction mixture is formed under inert gas (e.g., argon or nitrogen). In some embodiments, the fourth reaction mixture is stirred for about 2 hours at about 45° C. In some embodiments, the method further comprises recrystallizing compound 5. In some embodiments, compound 5 is recrystallized using DMSO, acetic acid, and water. In some embodiments, the cooled aqueous solution of sodium hydroxide and compound 5 is cooled to 0° C. to 7° C. In some embodiments, the cooled aqueous solution of sodium hydroxide and compound 5 is cooled to 4° C. to 5° C. In some embodiments, the fifth reaction mixture is formed under inert gas (e.g., argon or nitrogen). In some embodiments, the fifth reaction mixture is stirred for about 1 hour while cooled with an ice bath. In some embodiments, the fifth reaction mixture is stirred overnight (e.g., 8-15 hours) at room temperature. In some embodiments, the pH of the fifth reaction mixture is adjusted to pH of 8-9 using acetic acid. In some embodiments, the method further comprises recrystallizing compound A under methanolic conditions. In some embodiments, the methanolic conditions comprise, consist essentially of, or consist of methanol, triethylamine, and acetic acid.

In another aspect, provided herein is a method to prepare compound A:

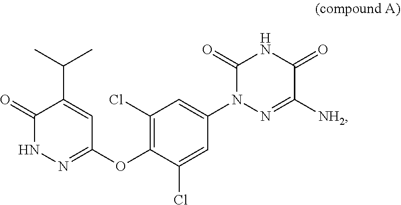 [SMILES: CC(C)c1cc(Oc2c(Cl)cc(-n3nc(N)c(=O)[nH]c3=O)cc2Cl)n[nH]c1=O]

wherein the method comprises:
   (1) adding acetic acid, water, and aqueous HCl to compound 1 and compound 2A-Et:

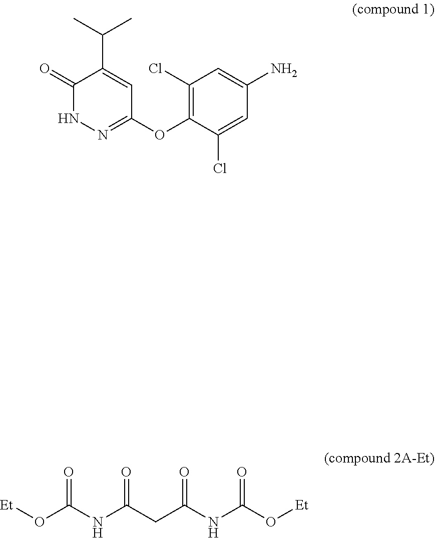 [SMILES: CC(C)c1cc(Oc2c(Cl)cc(N)cc2Cl)n[nH]c1=O ; CCOC(=O)NC(=O)CC(=O)NC(=O)OCC]

to form an initial reaction mixture;
   (2) adding an aqueous solution of sodium nitrite to the initial reaction mixture to form a second reaction mixture while maintaining the temperature at room temperature;
   (3) stirring the second reaction mixture at room temperature;
   (4) adding sodium acetate to the second reaction mixture while maintaining the temperature at no more than 30° C. to form a third reaction mixture;
   (5) stirring the third reaction mixture at room temperature;
   (6) isolating compound 3A-Et:

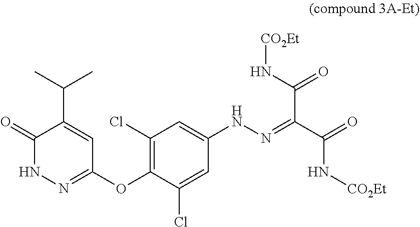 [SMILES: CCOC(=O)NC(=O)C(=NNc1cc(Cl)c(Oc2cc(C(C)C)c(=O)[nH]n2)c(Cl)c1)C(=O)NC(=O)OCC]

by filtration;
   (7) adding ammonia in methanol to compound 3A-Et and potassium carbonate to form a fourth reaction mixture;
   (8) stirring the fourth reaction mixture at about 45° C.;
   (9) adjusting the pH of the fourth reaction mixture to pH of 5-6 while maintaining the temperature at room temperature;
   (10) isolating compound 5:

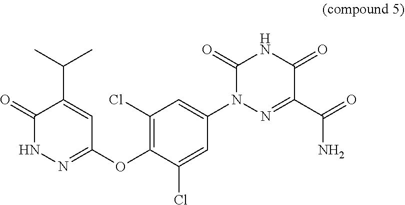 [SMILES: CC(C)c1cc(Oc2c(Cl)cc(-n3nc(C(N)=O)c(=O)[nH]c3=O)cc2Cl)n[nH]c1=O]

by filtration;
   (11) adding bleach to a cooled aqueous solution of sodium hydroxide and compound 5 to form a fifth reaction mixture;
   (12) stirring the fifth reaction mixture while cooled with an ice bath;
   (13) stirring the fifth reaction mixture at 35-45° C.;
   (14) adjusting pH of the fifth reaction mixture to pH of 5-6, with stirring, to form a slurry; and
   (15) isolating compound A from the slurry by filtration.
      In some embodiments, the initial reaction mixture is formed under inert gas (e.g., argon or nitrogen). In some embodiments, the second reaction mixture is stirred for about 30 minutes at room temperature. In some embodiments, the third reaction mixture is stirred overnight (e.g., 8-15 hours) at room temperature. In some embodiments, the fourth reaction mixture is formed under inert gas (e.g., argon or nitrogen). In some embodiments, the fourth reaction mixture is stirred for at least 18 hours at about 45° C. In some embodiments, the method further comprises triturating compound 5. In some embodiments, compound 5 is triturated using tetrahydrofuran (THF). In some embodiments, the cooled aqueous solution of sodium hydroxide and compound 5 is cooled to 4° C. to 5° C. In some embodiments, the fifth reaction mixture is formed under inert gas (e.g., argon or nitrogen). In some embodiments, the fifth reaction mixture is stirred for about 90 min while cooled with an ice bath. In some embodiments, the fifth reaction mixture is stirred for about 6 hours at 35-45° C. In some embodiments, the pH of the fifth reaction mixture is adjusted to pH of 5-6 using hydrochloric acid. In some embodiments, the method further comprises triturating compound A using acetic acid and a combination of THF and acetic acid.

In some embodiments, compound A is prepared according to Scheme 2.

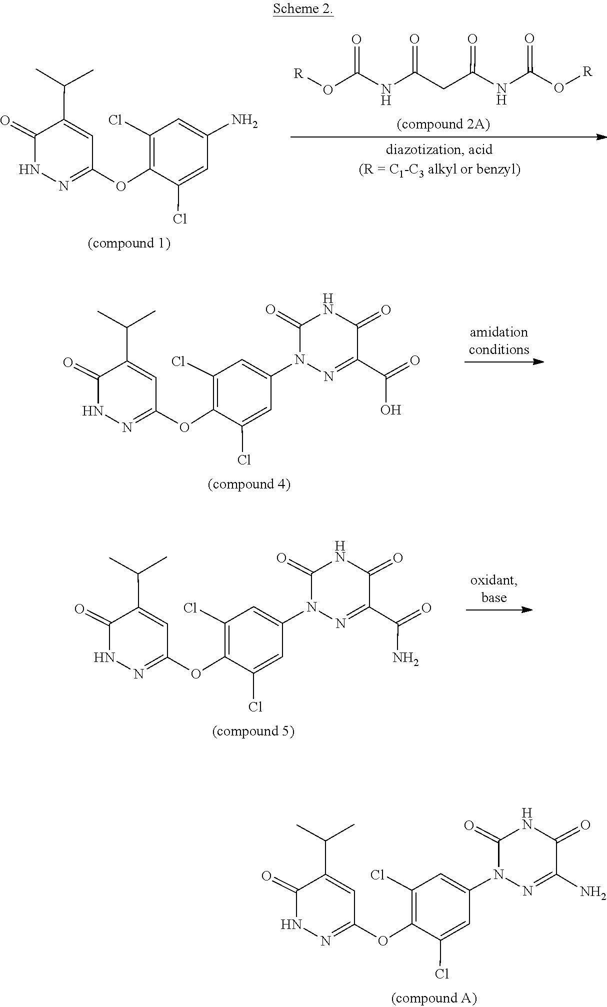

Accordingly, in some embodiments, the method to prepare compound A further comprises reacting compound 1:

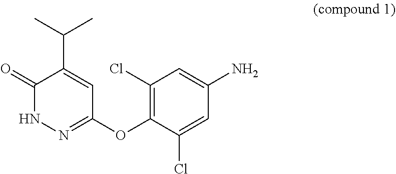 [SMILES: CC(C)c1cc(Oc2c(Cl)cc(N)cc2Cl)n[nH]c1=O]

with compound 2A:

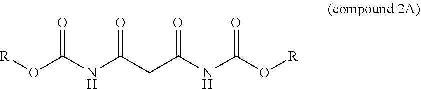

wherein R is C₁-C₃ alkyl or benzyl, under diazotization conditions followed by cyclizing under acidic conditions in a one-pot reaction to prepare compound 4:

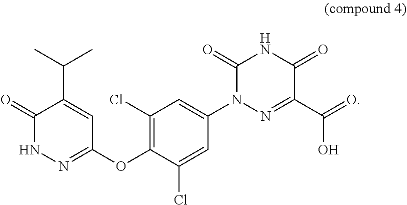 [SMILES: CC(C)c1cc(Oc2c(Cl)cc(-n3nc(C(=O)O)c(=O)[nH]c3=O)cc2Cl)n[nH]c1=O]

In some embodiments, the diazotization conditions comprise nitrite salt and one or more acids. In some embodiments, the diazotization conditions comprise sodium nitrite and hydrochloric acid. In some embodiments, the diazotization conditions are under inert gas (e.g., argon or nitrogen). In some embodiments, compound 1 in aqueous acetic acid is reacted with sodium nitrite and hydrochloric acid prior to reaction with compound 2A in the presence of sodium acetate. In some embodiments, the acidic conditions comprise aqueous hydrogen chloride. In some embodiments, the cyclizing is performed at about 105° C. In some embodiments, the method to prepare compound A further comprises reacting compound 4 under amidation conditions to prepare compound 5. In some embodiments, the amidation conditions comprise, consist essentially of, or consist of carbonyldiimidazole and NH₃ in dimethylformamide and water.

Accordingly, in another aspect, provided herein is a method to prepare compound A:

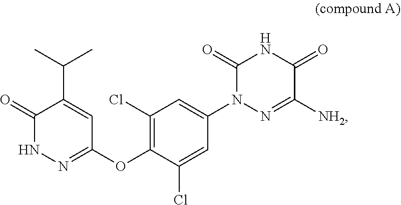 [SMILES: CC(C)c1cc(Oc2c(Cl)cc(-n3nc(N)c(=O)[nH]c3=O)cc2Cl)n[nH]c1=O]

wherein the method comprises:
   (1) adding an aqueous solution of sodium nitrite to a cooled solution of acetic acid, aqueous HCl, and compound 1:

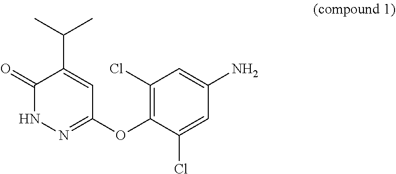 [SMILES: CC(C)c1cc(Oc2c(Cl)cc(N)cc2Cl)n[nH]c1=O]

to form an initial reaction mixture;
   (2) stirring the initial reaction mixture while cooled with an ice bath and then adding a solution of sodium acetate and compound 2A-Et:

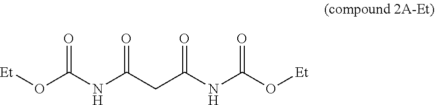 [SMILES: CCOC(=O)NC(=O)CC(=O)NC(=O)OCC]

to the initial reaction mixture to form a second reaction mixture while maintaining the cooling by ice bath;
   (3) stirring the second reaction mixture at room temperature;
   (4) adding sodium acetate to the second reaction mixture at room temperature to form a third reaction mixture;
   (5) stirring the third reaction mixture at about 80° C.;
   (6) stirring the third reaction mixture at room temperature;
   (7) adding aqueous hydrogen chloride to the third reaction mixture to form a fourth reaction mixture;
   (8) stirring the fourth reaction mixture at about 105° C.
   (9) isolating compound 4:

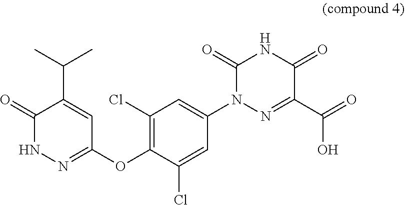 [SMILES: CC(C)c1cc(Oc2c(Cl)cc(-n3nc(C(=O)O)c(=O)[nH]c3=O)cc2Cl)n[nH]c1=O]

by filtration;
   (10) adding carbonyldiimidazole to a solution of compound 4 in dimethylformamide to form a fifth reaction mixture;
   (11) stirring the fifth reaction mixture at room temperature;
   (12) adding ammonia in water to the fifth reaction mixture to form a sixth reaction mixture;
   (13) stirring the sixth reaction mixture at room temperature;
   (14) adding aqueous hydrogen chloride to the sixth reaction mixture adjust pH to 7;
   (15) isolating compound 5:

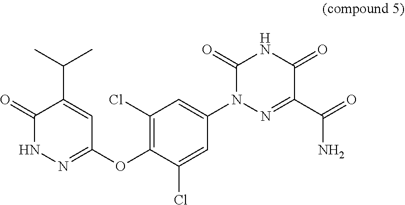 [SMILES: CC(C)c1cc(Oc2c(Cl)cc(-n3nc(C(N)=O)c(=O)[nH]c3=O)cc2Cl)n[nH]c1=O]

by filtration;
   (16) adding bleach to a cooled aqueous solution of sodium hydroxide and compound 5 to form a seventh reaction mixture;
   (17) stirring the seventh reaction mixture while cooled with an ice bath;
   (18) stirring the seventh reaction mixture at room temperature;
   (19) adjusting pH of the seventh reaction mixture to pH of 8-9, with stirring; and
   (20) isolating compound A by filtration.
      In some embodiments, the cooled solution of acetic acid, aqueous HCl, and compound 1 is at about 5° C. In some embodiments, the initial reaction mixture is stirred for at least 1 hour at about 5° C. In some embodiments, the initial reaction mixture is stirred for about 1 hour at about 5° C. In some embodiments, the second reaction mixture is stirred for at least 1 hour at room temperature. In some embodiments, the second reaction mixture is stirred for about 1 hour at room temperature. In some embodiments, the third reaction mixture is stirred for about 2 hours at about 80° C. In some embodiments, the third reaction mixture is stirred for 15 minutes at room temperature. In some embodiments, the fourth reaction mixture is stirred for about 2 hours at about 105° C. In some embodiments, the fifth reaction mixture is stirred for 30 minutes at room temperature. In some embodiments, the sixth reaction mixture is stirred for at least 1 hour at room temperature. In some embodiments, the sixth reaction mixture is stirred for about 1 hour at room temperature. In some embodiments, the method further comprises recrystallizing compound 5. In some embodiments, compound 5 is recrystallized using DMSO, acetic acid, and water. In some embodiments, the cooled aqueous solution of sodium hydroxide and compound 5 is cooled to 0° C. to 7° C. In some embodiments, the cooled aqueous solution of sodium hydroxide and compound 5 is cooled to 4° C. to 5° C. In some embodiments, the seventh reaction mixture is formed under inert gas (e.g., argon or nitrogen). In some embodiments, the seventh reaction mixture is stirred for about 1 hour while cooled with an ice bath. In some embodiments, the seventh reaction mixture is stirred overnight (e.g., 8-15 hours) at room temperature. In some embodiments, the pH of the seventh reaction mixture is adjusted to pH of 8-9 using acetic acid. In some embodiments, the method further comprises recrystallizing compound A under methanolic conditions. In some embodiments, the methanolic conditions comprise, consist essentially of, or consist of methanol, triethylamine, and acetic acid.

In some embodiments, compound A is prepared according to Scheme 3.

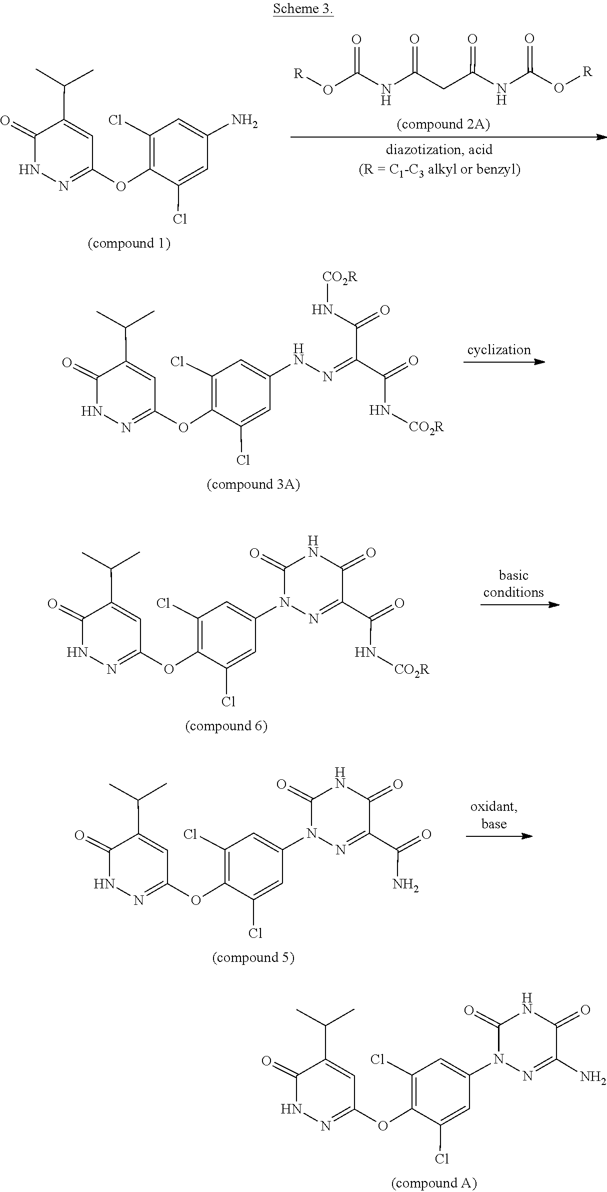

Accordingly, in some embodiments, the method to prepare compound A further comprises cyclizing compound 3A to prepare compound 6:

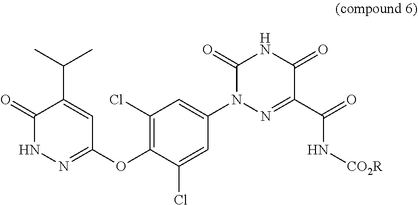

wherein R is C₁-C₃ alkyl or benzyl. In some embodiments, the cyclizing is under inert gas (e.g., argon or nitrogen). In some embodiments, the cyclizing is performed under refluxing buffered conditions. In some embodiments, the cyclizing is performed under buffered conditions at a temperature of about 100° C. to about 117° C., or a range therebetween. This includes a temperature of about 100, 101, 102, 103, 104, 105, 106, 107, 108, 109, 110, 111, 112, 113, 114, 115, 116, or 117° C., including any value therebetween. In some embodiments, the buffered conditions comprise, consist essentially of, or consist of aqueous acetic acid and acetate salt. In some embodiments, the buffered conditions comprise, consist essentially of, or consist of aqueous acetic acid and sodium acetate.

In some embodiments, the method to prepare compound A further comprises reacting compound 6 under basic conditions to prepare compound 5. In some embodiments, the basic conditions are under inert gas (e.g., argon or nitrogen). In some embodiments, the basic conditions comprise ammonia. In some embodiments, the basic conditions further comprise potassium carbonate. In some embodiments, the basic conditions comprise methanol as solvent. In some embodiments, compound 6 is heated under basic conditions to prepare compound 5. In some embodiments, compound 6 is heated at about 50° C. to about 60° C. under basic conditions to prepare compound 5. This includes about 50, 51, 52, 53, 54, 55, 56, 57, 58, 59, or 60° C., including any value therebetween. In some embodiments, compound 6 is heated at about 50° C. to about 60° C. under basic conditions to prepare compound 5, wherein the basic conditions comprise, consist essentially of, or consist of potassium carbonate and ammonia in methanol. In some embodiments, compound 6 is heated at about 50° C. to about 60° C. under basic conditions to prepare compound 5, wherein the basic conditions comprise, consist essentially of, or consist of ammonia in methanol.

Accordingly, in another aspect, provided herein is a method to prepare compound A:

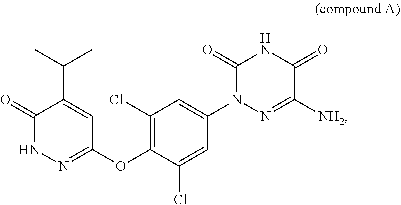 [SMILES: CC(C)c1cc(Oc2c(Cl)cc(-n3nc(N)c(=O)[nH]c3=O)cc2Cl)n[nH]c1=O]

wherein the method comprises:
   (1) adding acetic acid, water, and aqueous HCl to compound 1 and compound 2A-Et:

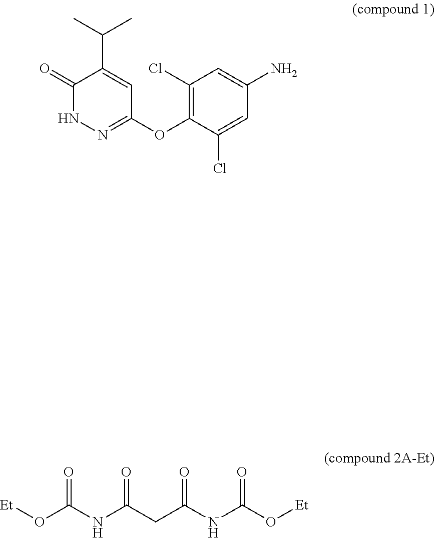 [SMILES: CC(C)c1cc(Oc2c(Cl)cc(N)cc2Cl)n[nH]c1=O ; CCOC(=O)NC(=O)CC(=O)NC(=O)OCC]

to form an initial reaction mixture;
   (2) cooling the initial reaction mixture with an ice bath and then adding an aqueous solution of sodium nitrite to the initial reaction mixture to form a second reaction mixture while maintaining the cooling by ice bath;
   (3) stirring the second reaction mixture while cooled with the ice bath;
   (4) adding sodium acetate to the second reaction mixture while cooled with the ice bath to form a third reaction mixture;
   (5) stirring the third reaction mixture while cooled with an ice bath;
   (6) stirring the third reaction mixture at room temperature;
   (7) isolating compound 3A-Et:

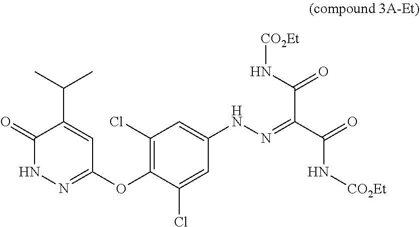 [SMILES: CCOC(=O)NC(=O)C(=NNc1cc(Cl)c(Oc2cc(C(C)C)c(=O)[nH]n2)c(Cl)c1)C(=O)NC(=O)OCC]

by filtration;
   (8) adding sodium acetate and acetic acid to compound 3A-Et to form a fourth reaction mixture;
   (9) heating the fourth reaction mixture at 117° C.;
   (10) cooling the fourth reaction mixture to room temperature before adding water;
   (11) isolating compound 6-Et:

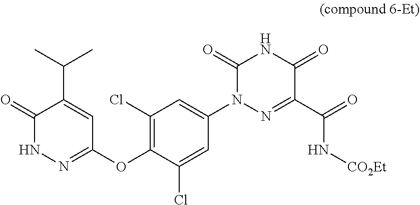 [SMILES: CCOC(=O)NC(=O)c1nn(-c2cc(Cl)c(Oc3cc(C(C)C)c(=O)[nH]n3)c(Cl)c2)c(=O)[nH]c1=O]

by filtration;
   (12) (a) adding ammonia in methanol to compound 6-Et and potassium carbonate to form a fifth reaction mixture; and stirring the fifth reaction mixture at about 55° C.; or
      (b) adding ammonia in methanol to compound 6-Et to form a sixth reaction mixture; stirring the sixth reaction mixture at about 55° C.; and then stirring the sixth reaction mixture at room temperature;
   (13) isolating compound 5:

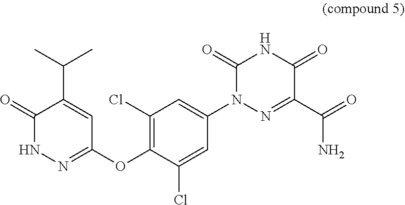 [SMILES: CC(C)c1cc(Oc2c(Cl)cc(-n3nc(C(N)=O)c(=O)[nH]c3=O)cc2Cl)n[nH]c1=O]

from the fifth reaction mixture or the sixth reaction mixture;
   (14) adding bleach to a cooled aqueous solution of sodium hydroxide and compound 5 to form a seventh reaction mixture;
   (15) stirring the seventh reaction mixture while cooled with an ice bath;
   (16) stirring the seventh reaction mixture at room temperature;
   (17) adjusting pH of the seventh reaction mixture to pH of 8-9, with stirring; and
   (18) isolating compound A by filtration.
      In some embodiments, the initial reaction mixture is formed under inert gas (e.g., argon or nitrogen). In some embodiments, the second reaction mixture is stirred for about 30 minutes while cooled with the ice bath. In some embodiments, the third reaction mixture is stirred for about 70 minutes while cooled with the ice bath. In some embodiments, the third reaction mixture is stirred overnight (e.g., 8-15 hours) at room temperature. In some embodiments, the fourth reaction mixture is formed under inert gas (e.g., argon or nitrogen). In some embodiments, the fourth reaction mixture is stirred for about 50 minutes at about 117° C. In some embodiments, the fifth reaction mixture is formed under inert gas (e.g., argon or nitrogen). In some embodiments, the fifth reaction mixture is stirred for about 3.5 hours at about 55° C. In some embodiments, the sixth reaction mixture is formed under inert gas (e.g., argon or nitrogen). In some embodiments, the sixth reaction mixture is stirred for about 3 hours at about 55° C. and then stirred for 50-60 hours at room temperature. In some embodiments, the cooled aqueous solution of sodium hydroxide and compound 5 is cooled to 0° C. to 7° C. In some embodiments, the cooled aqueous solution of sodium hydroxide and compound 5 is cooled to 4° C. to 5° C. In some embodiments, the seventh reaction mixture is formed under inert gas (e.g., argon or nitrogen). In some embodiments, the seventh reaction mixture is stirred for about 1 hour while cooled with an ice bath. In some embodiments, the seventh reaction mixture is stirred overnight (e.g., 8-15 hours) at room temperature. In some embodiments, the pH of the seventh reaction mixture is adjusted to pH of 8-9 using acetic acid. In some embodiments, the method further comprises recrystallizing compound A under methanolic conditions. In some embodiments, the methanolic conditions comprise, consist essentially of, or consist of methanol, triethylamine, and acetic acid.

In some embodiments, compound A is prepared according to Scheme 4.

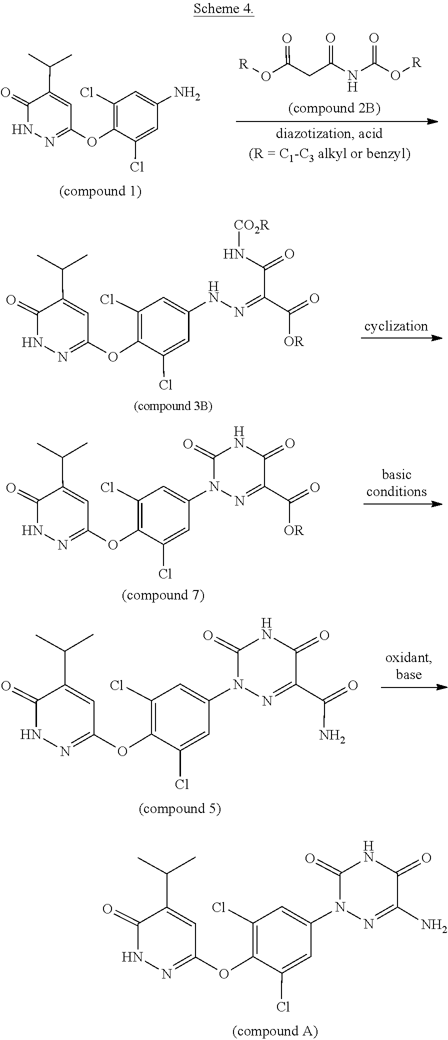

Accordingly, in some embodiments, the method to prepare compound A further comprises reacting compound 1:

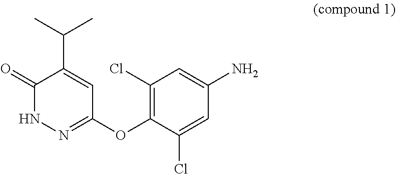 [SMILES: CC(C)c1cc(Oc2c(Cl)cc(N)cc2Cl)n[nH]c1=O]

with compound 2B:

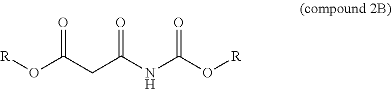

wherein R is C₁-C₃ alkyl or benzyl, under diazotization conditions to prepare compound 3B:

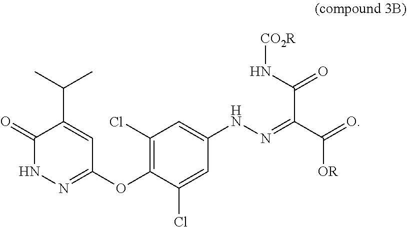

In some embodiments, R is C₁-C₃ alkyl. In some embodiments, R is methyl or ethyl. In some embodiments, R is ethyl. In some embodiments, R is methyl. In some embodiments, R is isopropyl. In some embodiments, R is benzyl.

In some embodiments, the diazotization conditions comprise nitrite salt and one or more acids. In some embodiments, the diazotization conditions comprise sodium nitrite and hydrochloric acid. In some embodiments, the diazotization conditions are under inert gas (e.g., argon or nitrogen). In some embodiments, compound 1 in aqueous acetic acid is reacted with compound 2B in the presence of sodium nitrite and hydrochloric acid prior to addition of sodium acetate to prepare compound 3B.

In some embodiments, the method to prepare compound A further comprises cyclizing compound 3B to prepare compound 7:

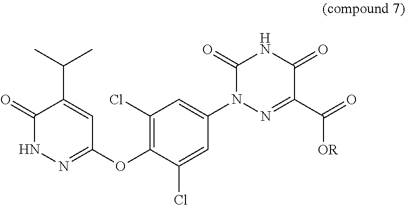

wherein R is C₁-C₃ alkyl or benzyl. In some embodiments, the cyclizing is under inert gas (e.g., argon or nitrogen). In some embodiments, the cyclizing is performed under refluxing buffered conditions. In some embodiments, the cyclizing is performed under buffered conditions at a temperature of about 100° C. to about 117° C., or a range therebetween. This includes a temperature of about 100, 101, 102, 103, 104, 105, 106, 107, 108, 109, 110, 111, 112, 113, 114, 115, 116, or 117° C., including any value therebetween. In some embodiments, the buffered conditions comprise, consist essentially of, or consist of aqueous acetic acid and acetate salt. In some embodiments, the buffered conditions comprise, consist essentially of, or consist of aqueous acetic acid and sodium acetate.

In some embodiments, the method to prepare compound A further comprises reacting compound 7 under basic conditions to prepare compound 5. In some embodiments, the basic conditions comprise ammonia. In some embodiments, the basic conditions comprise ammonia in methanol. In some embodiments, the basic conditions further comprise potassium carbonate. In some embodiments, the basic conditions comprise, consist essentially of, or consist of potassium carbonate and ammonia in methanol. In some embodiments, compound 7 is reacted under basic conditions at room temperature to prepare compound 5.

Accordingly, in another aspect, provided herein is a method to prepare compound A:

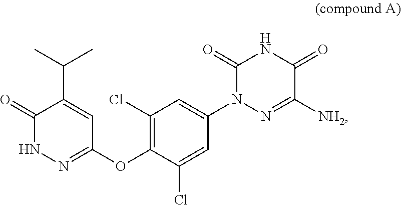 [SMILES: CC(C)c1cc(Oc2c(Cl)cc(-n3nc(N)c(=O)[nH]c3=O)cc2Cl)n[nH]c1=O]

wherein the method comprises:
   (1) adding acetic acid and water to compound 1 and compound 2B-Et:

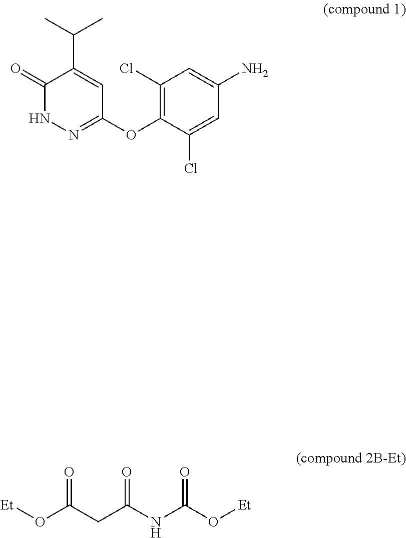 [SMILES: CC(C)c1cc(Oc2c(Cl)cc(N)cc2Cl)n[nH]c1=O ; CCOC(=O)CC(=O)NC(=O)OCC]

to form an initial reaction mixture;
   (2) cooling the initial reaction mixture with an ice bath and then adding aqueous HCl and an aqueous solution of sodium nitrite to the initial reaction mixture to form a second reaction mixture while maintaining the cooling by ice bath;
   (3) stirring the second reaction mixture while cooled with the ice bath;
   (4) adding sodium acetate to the second reaction mixture while cooled with the ice bath to form a third reaction mixture;
   (5) stirring the third reaction mixture while cooled with an ice bath;
   (6) stirring the third reaction mixture while warming to room temperature;
   (7) isolating compound 3B-Et:

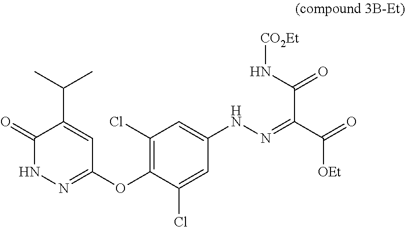 [SMILES: CCOC(=O)NC(=O)/C(=N\Nc1cc(Cl)c(Oc2cc(C(C)C)c(=O)[nH]n2)c(Cl)c1)C(=O)OCC]

by filtration;
   (8) adding sodium acetate and acetic acid to compound 3B-Et to form a fourth reaction mixture;
   (9) heating the fourth reaction mixture to reflux (i.e., about 117° C.);
   (10) cooling the fourth reaction mixture to room temperature before adding water;
   (11) isolating compound 7-Et:

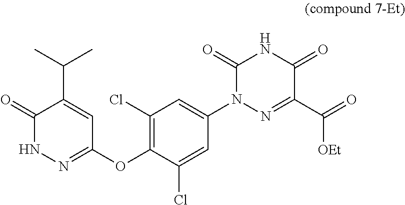 [SMILES: CCOC(=O)c1nn(-c2cc(Cl)c(Oc3cc(C(C)C)c(=O)[nH]n3)c(Cl)c2)c(=O)[nH]c1=O]

by filtration;
   (12) adding ammonia in methanol to compound 7-Et to form a fifth reaction mixture;
   (13) stirring the fifth reaction mixture at room temperature;
   (14) isolating compound 5:

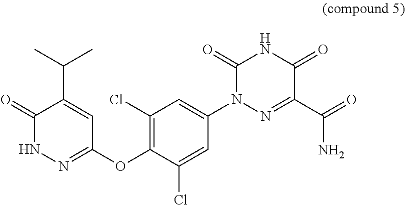 [SMILES: CC(C)c1cc(Oc2c(Cl)cc(-n3nc(C(N)=O)c(=O)[nH]c3=O)cc2Cl)n[nH]c1=O]

from the fifth reaction mixture;
   (15) adding bleach to a cooled aqueous solution of sodium hydroxide and compound 5 to form a sixth reaction mixture;
   (16) stirring the sixth reaction mixture while cooled with an ice bath;
   (17) stirring the sixth reaction mixture at room temperature;
   (18) adjusting pH of the sixth reaction mixture to pH of 8-9, with stirring; and
   (19) isolating compound A by filtration.
      In some embodiments, the initial reaction mixture is formed under inert gas (e.g., argon or nitrogen). In some embodiments, the second reaction mixture is stirred for about 30 minutes while cooled with the ice bath. In some embodiments, the third reaction mixture is stirred for about 60 minutes while cooled with the ice bath. In some embodiments, the fourth reaction mixture is formed under inert gas (e.g., argon or nitrogen). In some embodiments, the fourth reaction mixture is stirred at reflux for about 2 hours and 15 minutes. In some embodiments, the fourth reaction mixture is stirred for about 2 hours and 15 minutes at about 117° C. In some embodiments, the fifth reaction mixture is stirred for 50-60 hours at room temperature. In some embodiments, the fifth reaction mixture is stirred for about 55 hours at room temperature. In some embodiments, the cooled aqueous solution of sodium hydroxide and compound 5 is cooled to 0° C. to 7° C. In some embodiments, the cooled aqueous solution of sodium hydroxide and compound 5 is cooled to 4° C. to 5° C. In some embodiments, the sixth reaction mixture is formed under inert gas (e.g., argon or nitrogen). In some embodiments, the sixth reaction mixture is stirred for about 1 hour while cooled with an ice bath. In some embodiments, the sixth reaction mixture is stirred overnight (e.g., 8-15 hours) at room temperature. In some embodiments, the pH of the sixth reaction mixture is adjusted to pH of 8-9 using acetic acid. In some embodiments, the method further comprises recrystallizing compound A under methanolic conditions. In some embodiments, the methanolic conditions comprise, consist essentially of, or consist of methanol, triethylamine, and acetic acid.

In some embodiments, compound A is prepared according to Scheme 5.

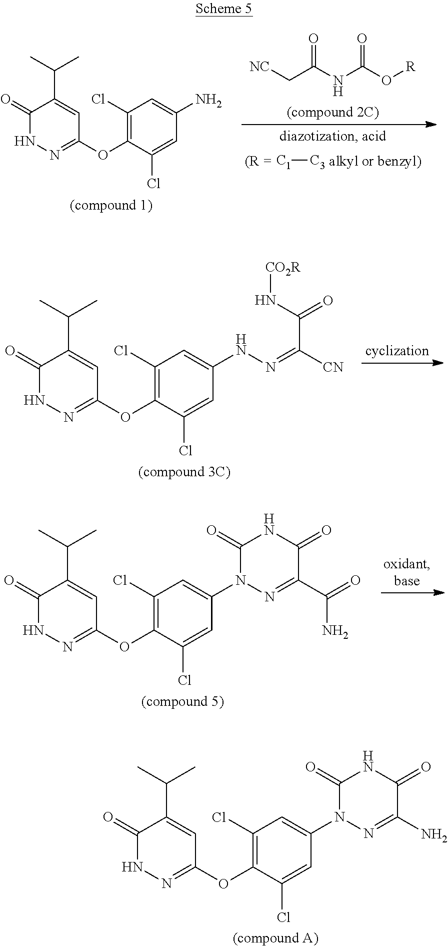

Accordingly, in some embodiments, the method to prepare compound A further comprises cyclizing compound 3C:

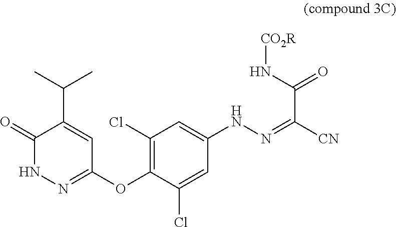

wherein R is C₁-C₃ alkyl or benzyl, to prepare compound 5 in a one-pot step. In some embodiments, the one-pot step is under inert gas (e.g., argon or nitrogen). In some embodiments, the one-pot step comprises, consists essentially of, or consists of refluxing buffered conditions, followed by heating under acidic conditions. In some embodiments, the buffered conditions comprise, consist essentially of, or consist of aqueous acetic acid and acetate salt. In some embodiments, the buffered conditions comprise, consist essentially of, or consist of aqueous acetic acid and sodium acetate. Accordingly, reflux temperature is about 117° C. for buffered conditions comprising aqueous acetic acid and sodium acetate. In some embodiments, the acidic conditions comprise trifluoracetic acid and sulfuric acid. In some embodiments, the heating under acidic conditions is conducted at about 80° C.

Accordingly, in another aspect, provided herein is a method to prepare compound A:

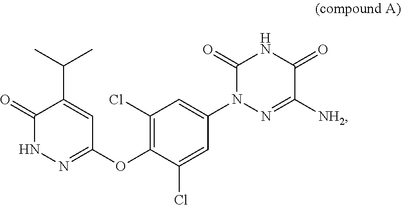 [SMILES: CC(C)c1cc(Oc2c(Cl)cc(-n3nc(N)c(=O)[nH]c3=O)cc2Cl)n[nH]c1=O]

wherein the method comprises:
   (1) adding sodium acetate and acetic acid to compound 3C-Et:

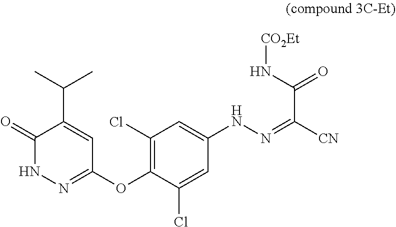 [SMILES: CCOC(=O)NC(=O)/C(C#N)=N\Nc1cc(Cl)c(Oc2cc(C(C)C)c(=O)[nH]n2)c(Cl)c1]

to form an initial reaction mixture;
   (2) heating the initial reaction mixture to reflux (i.e., about 117° C.);
   (3) cooling the initial reaction mixture to about 30° C. with a cool water bath before adding trifluoroacetic acid (TFA) and sulfuric acid to form a second reaction mixture while maintaining the cool water bath;
   (4) heating the second reaction mixture at about 80° C.;
   (5) cooling the second reaction mixture using an ice bath before adding water;
   (6) isolating compound 5:

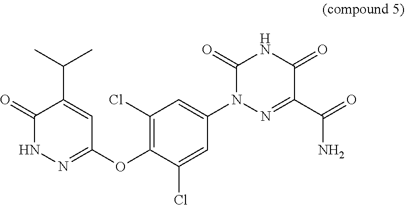 [SMILES: CC(C)c1cc(Oc2c(Cl)cc(-n3nc(C(N)=O)c(=O)[nH]c3=O)cc2Cl)n[nH]c1=O]

by filtration.
   (7) adding bleach to a cooled aqueous solution of sodium hydroxide and compound 5 to form a third reaction mixture;
   (8) stirring the third reaction mixture while cooled with an ice bath;
   (9) stirring the third reaction mixture at room temperature;
   (10) adjusting pH of the third reaction mixture to pH of 8-9, with stirring; and
   (11) isolating compound A by filtration.
      In some embodiments, the initial reaction mixture is formed under inert gas (e.g., argon or nitrogen). In some embodiments, the initial reaction mixture is stirred at reflux for about 70 minutes. In some embodiments, the initial reaction mixture is stirred for about 70 minutes at about 117° C. In some embodiments, the second reaction mixture is stirred for about 2 hours at about 80° C. In some embodiments, the method further comprises recrystallizing compound 5. In some embodiments, compound 5 is recrystallized using methanol, triethylamine, and acetic acid. In some embodiments, the cooled aqueous solution of sodium hydroxide and compound 5 is cooled to 0° C. to 7° C. In some embodiments, the cooled aqueous solution of sodium hydroxide and compound 5 is cooled to 4° C. to 5° C. In some embodiments, the third reaction mixture is formed under inert gas (e.g., argon or nitrogen). In some embodiments, the third reaction mixture is stirred for about 1 hour while cooled with an ice bath. In some embodiments, the third reaction mixture is stirred overnight (e.g., 8-15 hours) at room temperature. In some embodiments, the pH of the third reaction mixture is adjusted to pH of 8-9 using acetic acid. In some embodiments, the method further comprises recrystallizing compound A under methanolic conditions. In some embodiments, the methanolic conditions comprise, consist essentially of, or consist of methanol, triethylamine, and acetic acid.

In another aspect, provided herein is a compound having the structure of

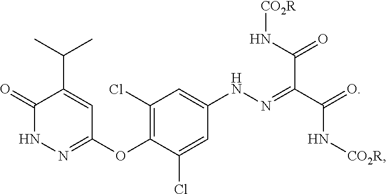

wherein R is C₁-C₃ alkyl or benzyl. In some embodiments, R is C₁-C₃ alkyl. In some embodiments, R is ethyl.

In another aspect, provided herein is a method to prepare compound 3A:

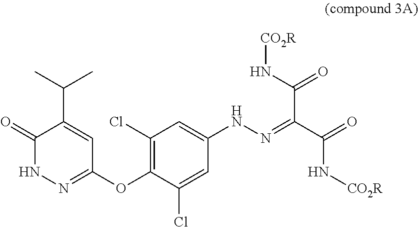

wherein the method comprises reacting compound 1:

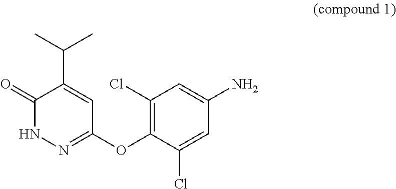 [SMILES: CC(C)c1cc(Oc2c(Cl)cc(N)cc2Cl)n[nH]c1=O]

with compound 2A:

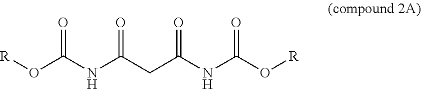

wherein R is C₁-C₃ alkyl or benzyl, under diazotization conditions to prepare compound 3A.

In another aspect, provided herein is a compound having the structure of

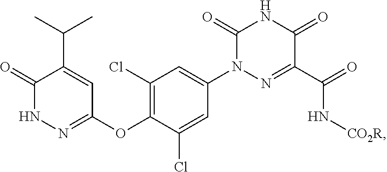

wherein R is C₁-C₃ alkyl or benzyl. In some embodiments, R is C₁-C₃ alkyl. In some embodiments, R is ethyl.

In another aspect, provided herein is a method to prepare compound 6:

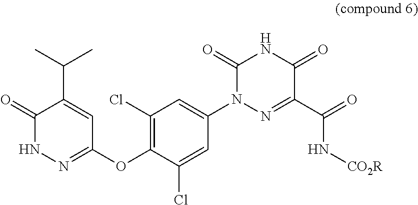

wherein the method comprises cyclizing compound 3A:

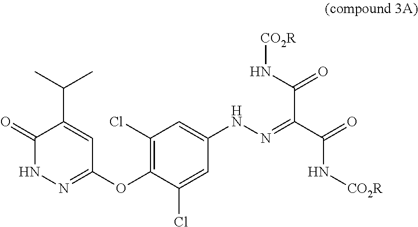

to prepare compound 6, wherein R is C₁-C₃ alkyl or benzyl.

In another aspect, provided herein is a compound having the structure of

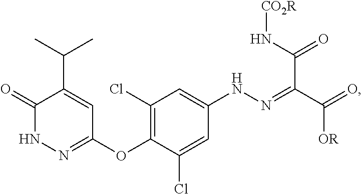

wherein R is C₁-C₃ alkyl or benzyl. In some embodiments, R is C₁-C₃ alkyl. In some embodiments, R is ethyl.

In another aspect, provided herein is a method to prepare compound 3B:

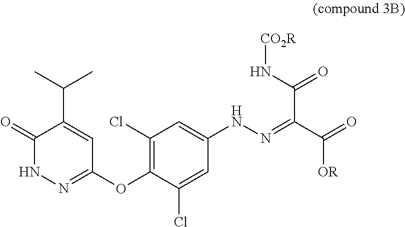

wherein the method comprises reacting compound 1:

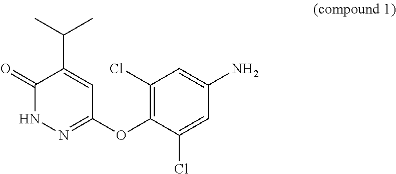 [SMILES: CC(C)c1cc(Oc2c(Cl)cc(N)cc2Cl)n[nH]c1=O]

with compound 2B:

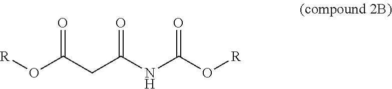

wherein R is C₁-C₃ alkyl or benzyl, under diazotization conditions to prepare compound 3B.

In another aspect, provided herein is a compound having the structure of

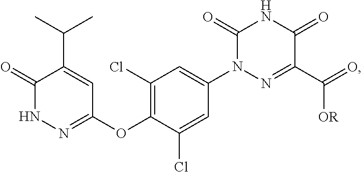

wherein R is C₁-C₃ alkyl or benzyl. In some embodiments, R is C₁-C₃ alkyl. In some embodiments, R is ethyl.

In another aspect, provided herein is a method to prepare compound 7:

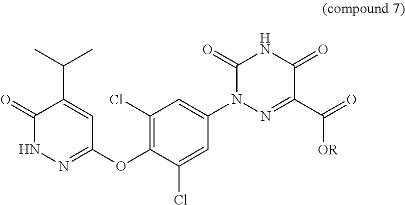

wherein the method comprises cyclizing compound 3B:

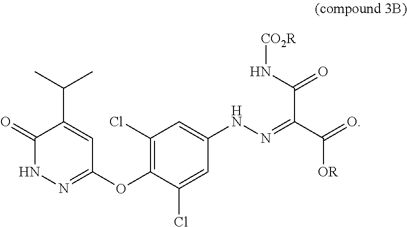

to prepare compound 7, wherein R is C₁-C₃ alkyl or benzyl.

### EXAMPLES

### Example 1. Preparation of Compound A

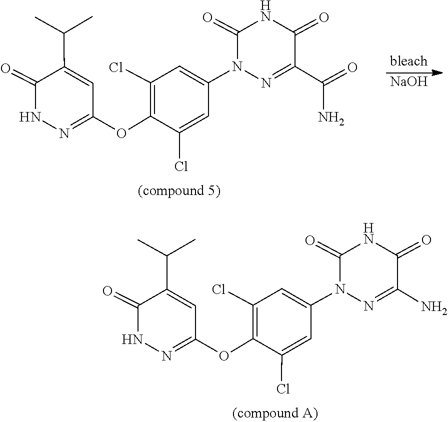

2-(3,5-Dichloro-4-((5-isopropyl-6-oxo-1,6-dihydropyridazin-3-yl)oxy)phenyl)-3,5-dioxo-2,3,4,5-tetrahydro-1,2,4-triazine-6-carboxamide (compound 5) (17.64 g) was charged to a reactor under argon. A solution of sodium hydroxide (5.45 g, 3.5 equiv.) in water (196 mL) was prepared, cooled to room temperature, and added to the reactor containing compound 5. The resultant slurry was stirred for 10 minutes, dissolving most solids. The red solution was cooled to 4-5° C., where a bleach solution (sodium hypochlorite, 11-15% available chlorine) (40.90 g, 1.55-2.12 equiv.) was added dropwise, causing the solution to change from red to yellow. This mixture was stirred for 1 hour with ice-bath cooling, then at room temperature overnight. After stirring at room temperature overnight, the reaction was judged to be complete by HPLC-MS. Acetic acid (3.6 g, 1.54 equiv.) was added, generating a slurry with pH of 8-9. The mixture was stirred for 70 minutes, and then the product was collected by filtration. The filter cake was washed with water and dried under vacuum at 65° C. to afford 13.85 g of crude compound A. This crude material underwent two recrystallizations as described in more detail below.

First recrystallization: crude compound A (8.26 g) was combined with methanol (230 mL) and stirred under argon. Triethylamine (5.90 g, 3.0 equiv.) was added, and the mixture was immersed in a 25° C.-water bath for 25 minutes, dissolving most solids. The solution was filtered through celite, removing a small amount of gray solid. The filtrate was stirred and acetic acid (4.67 g, 4 equiv.) was added over 1 minute, causing a recrystallization that began another minute later. After 25 minutes, water (33 mL) was added dropwise over 7 minutes. Ten minutes later, the slurry was cooled with an ice-water bath for 1 hour. The slurry was filtered, washed with ice-cold 3:1 methanol:water (40 mL), and dried under vacuum at 60° C. to furnish once-recrystallized compound A (5.92 g).

Second recrystallization: the once-recrystallized compound A (5.92 g) was mixed with methanol (148 mL) and water (21 mL). The resulting slurry was stirred, triethylamine (4.23 g, 3.0 equiv.) was added, and stirring was continued for 5 minutes to dissolve all solids. Acetic acid (2.79 g, 3.3 equiv.) was added dropwise, causing recrystallization. The slurry was stirred for 15 minutes at room temperature then for 45 minutes in an ice-water bath. The slurry was filtered, washed with ice-cold 3:1 methanol:water (35 mL), then dried at 65° C. under vacuum to afford compound A (5.52 g, 56% overall yield from compound 5). Purity=98.6% [254 nm]. ¹H NMR (d₆-DMSO): 12.27 (s, 1H), 12.21 (s, 1H), 7.85 (s, 2H), 7.43 (s, 1H), 6.54 (s, 2H), 3.07 (m, 1H), 1.19 (d, 6H); LCMS (ESI, m/z): 425 [M+H]⁺.

### Example 2. Preparation of 2-(3,5-dichloro-4-((5-isopropyl-6-oxo-1,6-dihydropyridazin-3-yl)oxy)phenyl)-3,5-dioxo-2,3,4,5-tetrahydro-1,2,4-triazine-6-carboxamide (compound 5)—Option 1

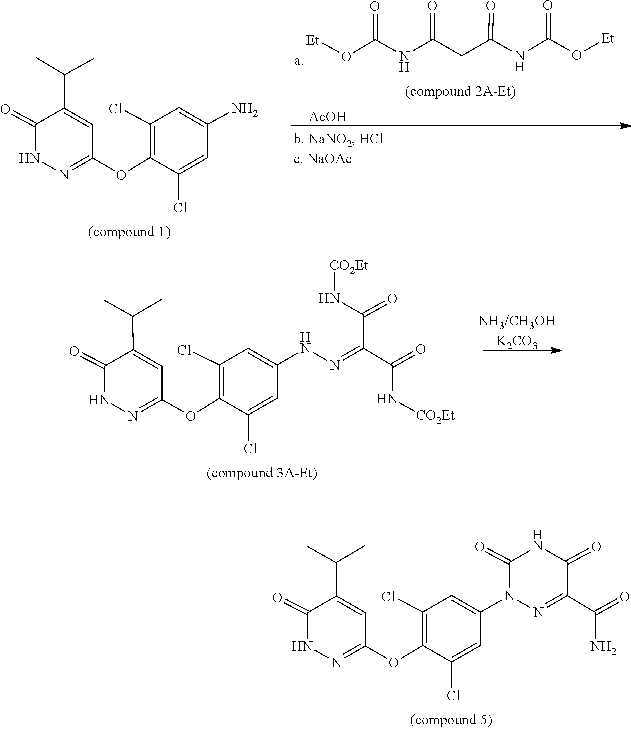 [SMILES: "AcOH" ; CCOC(=O)NC(=O)CC(=O)NC(=O)OCC ; CC(C)c1cc(Oc2c(Cl)cc(N)cc2Cl)n[nH]c1=O ; CCOC(=O)NC(=O)C(=NNc1cc(Cl)c(Oc2cc(C(C)C)c(=O)[nH]n2)c(Cl)c1)C(=O)NC(=O)OCC ; CC(C)c1cc(Oc2c(Cl)cc(-n3nc(C(N)=O)c(=O)[nH]c3=O)cc2Cl)n[nH]c1=O]

6-(4-Amino-2,6-dichlorophenoxy)-4-isopropylpyridazin-3(2H)-one (compound 1) was prepared as described in WO 2020/227549.

Step 1. Compound 1 (10.33 g, 1.00 equiv.) was combined with diethyl malonyldicarbamate (compound 2A-Et) (8.10 g, 1.00 equiv.) in a reactor and an argon atmosphere was established within. Acetic acid (155 mL) was added followed by water (103 mL) with stirring, creating a mostly homogeneous solution. Concentrated aqueous hydrogen chloride (˜12 M, 8.2-8.3 mL, ˜3 equiv.) was added, and the resulting homogeneous solution was immediately cooled in an ice bath. A solution of sodium nitrite (2.38 g, 1.05 equiv.) in water (7 mL) was dripped in so that T=4.6-4.9° C.—a slurry began forming during the latter half of the addition. After 30 minutes, a solution of sodium acetate trihydrate (14.42 g, 3.00 equiv.) was added dropwise at 1-4° C., causing solids to coagulate. The mixture was stirred for 70 minutes with ice-bath cooling and then allowed to warm to room temperature overnight with stirring. The resultant slurry was filtered through a fine-porosity fritted filter funnel over 20 minutes. The filter cake was washed with 1:1 acetic acid:water (50 mL) then dried at 70° C. in a vacuum oven to provide diethyl (2-(2-(3,5-dichloro-4-((5-isopropyl-6-oxo-1,6-dihydropyridazin-3-yl)oxy)phenyl)hydrazineylidene) malonyl)dicarbamate (compound 3A-Et) (19.30 g). LCMS (ESI, m/z): 571 [M+H]⁺.

Step 1 (kilogram scale). Compound 1 (8.8 kg, 1.0 equiv.) was combined with diethyl malonyldicarbamate (compound 2A-Et) (1.0 equiv.) in acetic acid (15 v/w) in a reactor under a nitrogen atmosphere at room temperature (25±5° C.). Water (10 v/w) was added to the reactor with stirring, followed by the addition of HCl (12 M, 3.0 equiv.). A solution of sodium nitrite (1.05 equiv.) in water (0.5 v/w) was added dropwise within 10 min to the reactor, maintaining a reaction mixture temperature of 25±5° C. The resultant mixture was further stirred at 25±5° C. for 30 min. A solution of sodium acetate (3.0 equiv.) in water (3.0 v/w) was then added dropwise to the reactor while maintaining the temperature to be no more than 30° C. before stirring the mixture overnight at 25±5° C. Reaction completion was observed by HPLC. Solids were filtered and washed with acetic acid/water (1:1, 5 v/w). The filter cake was dried at 50±5° C. for 18 hours to provide diethyl (2-(2-(3,5-dichloro-4-((5-isopropyl-6-oxo-1,6-dihydropyridazin-3-yl)oxy)-phenyl)hydrazineylidene) malonyl)dicarbamate (compound 3A-Et) (14.9 kg).

Step 2. Compound 3A-Et (4.54 g, 1.0 equiv.) was combined with potassium carbonate (2.20 g; 2.0 equiv.) in a reactor and an argon atmosphere was established, with the reactor open to a bubbler. 7 N NH₃ in methanol (91 mL, 20 volumes) was added, and the resulting slurry was stirred and heated to 45° C. for 2 hours, creating a homogeneous solution. A sample was withdrawn, mixed with 5% aq. Hydrogen chloride then DMSO, and analyzed by HPLC-MS, indicating reaction completion. The solution was cooled to room temperature and minimal water (22 mL) was added dropwise to initiate crystallization. After 15 minutes, additional water (6 mL) was added dropwise to the resulting slurry, which was then cooled using an ice bath. After 1 hour of cooling, the slurry was filtered through a medium-porosity fritted filter, and then the filter cake was washed three times with 15-mL portions of water. The filter cake was loaded into a round-bottomed flask, DMSO (42 mL) was added, and the mixture was stirred to dissolve the solids. Acetic acid (0.89 g, 2.0 equiv.) was added. The solution was cooled with a room temperature water bath while minimal water was added dropwise to initiate crystallization (12 mL). After 15 minutes, additional water (12 mL) was added dropwise. After 25 minutes of additional stirring, the slurry was cooled with an ice-water bath for 30 minutes before being filtered through a medium-porosity fritted filter funnel. The filter cake was washed four times with water then dried at 75° C. in a vacuum oven to provide compound 5 (2.35 g). LCMS (ESI, m/z): 453 [M+H]⁺. ¹H NMR (d₆-DMSO): 12.76 (s, 1H), 12.23 (s, 1H), 8.09 (s, 1H), 7.91 (s, 1H), 7.86 (s, 2H), 7.44 (s, 1H), 3.08 (m, 1H), 1.20 (s, 6H).

Step 2 (kilogram scale). Compound 3A-Et (14.0 kg, 1.0 equiv.) was combined with potassium carbonate (2.0 equiv.) in a reactor with 7 N NH₃ in methanol (15.0 v/w) at room temperature (25±5° C.). The reaction mixture was heated to 45±5° C. for at least 18 hours. Once reaction was shown to be complete by HPLC, the mixture was concentrated to 5-6 v/w while heating to about 45±5° C. After cooling to room temperature, HCl (2 M) was added to adjust pH to 5-6 while maintaining a temperature of 25±5° C. Solids were filtered and washed twice with water (2.0 v/w). Purity was assessed by HPLC. If purity was less than 98%, the solids were suspended in THF, heated to 45±5° C. for at least 3 hours, and then cooled to room temperature prior to filtration and washing with additional THF (2.0 v/w). If purity remained below 98%, the trituration with THF was repeated. Otherwise, the filter cake was dried at 45±5° C. for at least 12 hours to provide compound 5 (11.0 kg).

### Example 3. Preparation of 2-(3,5-dichloro-4-((5-isopropyl-6-oxo-1,6-dihydropyridazin-3-yl)oxy)phenyl)-3,5-dioxo-2,3,4,5-tetrahydro-1,2,4-triazine-6-carboxamide (compound 5)—Option 2

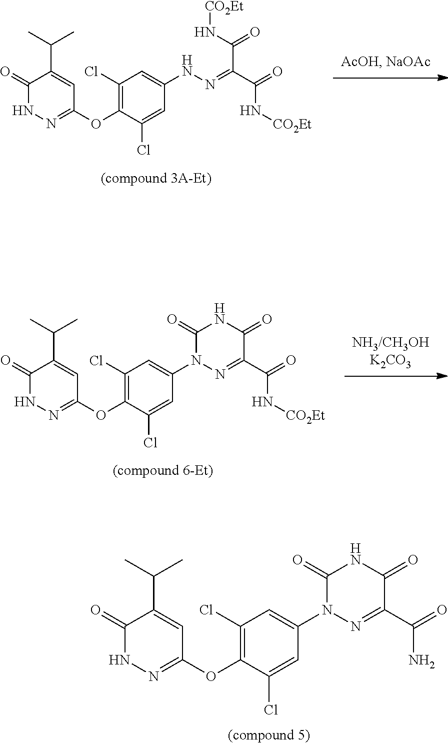 [SMILES: CCOC(=O)NC(=O)C(=NNc1cc(Cl)c(Oc2cc(C(C)C)c(=O)[nH]n2)c(Cl)c1)C(=O)NC(=O)OCC ; CCOC(=O)NC(=O)c1nn(-c2cc(Cl)c(Oc3cc(C(C)C)c(=O)[nH]n3)c(Cl)c2)c(=O)[nH]c1=O ; CC(C)c1cc(Oc2c(Cl)cc(-n3nc(C(N)=O)c(=O)[nH]c3=O)cc2Cl)n[nH]c1=O]

Step 1. Compound 3A-Et (10.39 g) was combined with sodium acetate (4.48 g) and acetic acid (105 mL) and stirred and heated under argon to 117° C. for 50 minutes. The mixture was cooled prior to dropwise addition of water (85 mL), initiating crystallization. The resulting slurry was stirred for 1 hour and filtered. The solids were washed with 2:1 H₂O:AcOH and then with H₂O alone prior to drying at 75° C. under vacuum to provide ethyl (2-(3,5-dichloro-4-((5-isopropyl-6-oxo-1,6-dihydropyridazin-3-yl)oxy)phenyl)-3,5-dioxo-2,3,4,5-tetrahydro-1,2,4-triazine-6-carbonyl)carbamate (compound 6-Et) (7.75 g). ¹H NMR (d₆-DMSO): 12.90 (s, 1H), 12.21 (s, 1H), 11.51 (s, 1H), 7.83 (s, 2H), 7.43 (s, 1H), 4.15 (q, 2H), 3.05 (m, 1H), 1.20 (m, 9H). LCMS (ESI, m/z): 525 [M+H]⁺.

Step 2. Compound 6-Et (0.34 g), potassium carbonate (108 mg, ˜1 equiv.), and a stir bar were combined under argon in a pressure tube. 7 N NH₃ in methanol (3.4 mL) was added, and the tube was sealed. The sealed tube was immersed in an oil bath which was then heated to 55° C. for 2 hours. At this point, the mixture remained a slurry. The tube was cooled, opened, and sampled. The sample was mixed with 5% aq. HCl then DMSO and analyzed by HPLC-MS, which indicated that reaction conversion was only 50% complete. More potassium carbonate (100 mg) and 7 N NH₃ in methanol (3.4 mL) were added to the tube, which was then re-sealed, and heating was continued. After 30 minutes, only a small amount of solids remained. After another hour, HPLC-MS indicated that the reaction was complete, with the major impurity being the analogous carboxylic acid. The slurry was filtered, and the filtrate was concentrated to a red oil. Methanol (9 mL) was added and, with stirring, the oil dissolved. Acetic acid (117 mg, 3 equiv.) was diluted with methanol (˜0.5 mL) and added dropwise, initiating crystallization. More acetic acid (˜3 drops) was added to cause pH to drop from 7 to 5. The resulting slurry was stirred 20 minutes, filtered, washed with methanol, and dried. This was the primary material. Secondary material was obtained by adding water to the filtrate until it was approximately 50% aqueous. After aging for 20 minutes, solids were collected by filtration and washed with 1:1 methanol:water. Both the primary and secondary material were dried at 70° C. under vacuum, providing 170 mg and 80 mg portions, respectively. However, only the secondary material had the desired ¹H NMR spectra. The primary material was dissolved in 7 N NH₃-methanol (4 mL) and concentrated. The resulting solid did not dissolve in methanol (5 mL) nor when triethylamine (114 mg, 3 equiv.) was added. The addition of water (2 drops), however, dissolved the solids completely. Acetic acid (211 mg) was diluted with water (3 mL) and added dropwise with stirring to the solution of the primary material, initiating a crystallization (pH=5). The slurry was stirred for 10 minutes, filtered, washed with 1:1 methanol:water, and dried at 70° C. under vacuum to afford compound 5 (130 mg). This material had the desired ¹H NMR spectra when analyzed in DMSO-d₆.

### Example 4. Preparation of 2-(3,5-dichloro-4-((5-isopropyl-6-oxo-1,6-dihydropyridazin-3-yl)oxy)phenyl)-3,5-dioxo-2,3,4,5-tetrahydro-1,2,4-triazine-6-carboxamide (compound 5)—Option 3

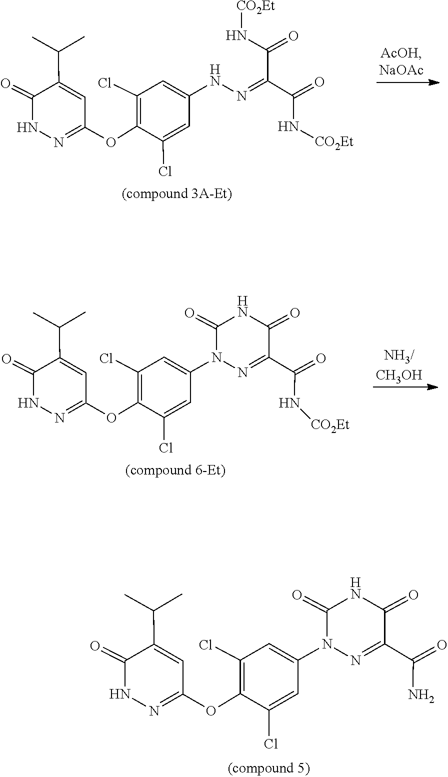 [SMILES: CCOC(=O)NC(=O)C(=NNc1cc(Cl)c(Oc2cc(C(C)C)c(=O)[nH]n2)c(Cl)c1)C(=O)NC(=O)OCC ; CCOC(=O)NC(=O)c1nn(-c2cc(Cl)c(Oc3cc(C(C)C)c(=O)[nH]n3)c(Cl)c2)c(=O)[nH]c1=O ; CC(C)c1cc(Oc2c(Cl)cc(-n3nc(C(N)=O)c(=O)[nH]c3=O)cc2Cl)n[nH]c1=O]

Step 1. Ethyl (2-(3,5-dichloro-4-((5-isopropyl-6-oxo-1,6-dihydropyridazin-3-yl)oxy)phenyl)-3,5-dioxo-2,3,4,5-tetrahydro-1,2,4-triazine-6-carbonyl)carbamate (compound 6-Et) was prepared as described in Example 3, Step 1.

Step 2. Compound 6-Et (164 mg) was mixed with 7 N NH₃ in methanol (2 mL) in a sealed vial. [HPLC-MS samples were prepared throughout the process by mixing samples with 5% aq. HCl then diluting with DMSO] The resulting slurry was heated in a 55° C. mantle for 3 hours [HPLC-MS at this time showed ˜50% conversion] then stirred over a weekend at room temperature. The solution was syringe-filtered away from a small amount of solids, and the filtrate was concentrated. Methanol (1.5 mL) was added to the concentrate followed by triethylamine (97 mg, 3 equiv.). The homogeneous solution was stirred while acetic acid (77 mg, 4 equiv.) was added dropwise. However, the pH of this mixture was only at 6-7, so the pH of this mixture was further adjusted to pH 5 through dropwise addition of acetic acid. Crystallization began within 30 seconds. After stirring for 0.5 h, the slurry was filtered and washed with methanol. The resultant solid was dried at 70° C. under vacuum to provide compound 5 (100 mg), as confirmed by LCMS and ¹H NMR.

### Example 5. Preparation of 2-(3,5-dichloro-4-((5-isopropyl-6-oxo-1,6-dihydropyridazin-3-yl)oxy)phenyl)-3,5-dioxo-2,3,4,5-tetrahydro-1,2,4-triazine-6-carboxamide (compound 5)—Option 4

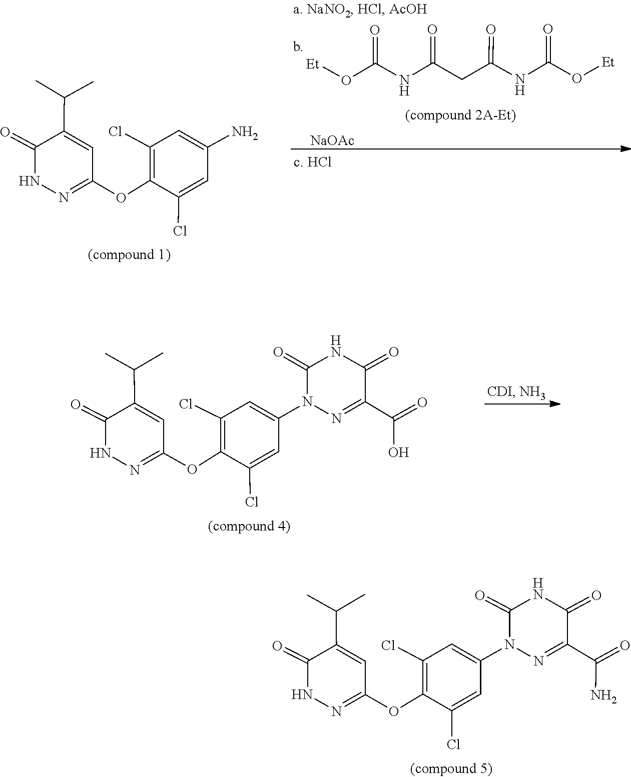 [SMILES: CCOC(=O)NC(=O)CC(=O)NC(=O)OCC ; CC(C)c1cc(Oc2c(Cl)cc(N)cc2Cl)n[nH]c1=O ; CC(C)c1cc(Oc2c(Cl)cc(-n3nc(C(=O)O)c(=O)[nH]c3=O)cc2Cl)n[nH]c1=O ; "NaOAc" ; CC(C)c1cc(Oc2c(Cl)cc(-n3nc(C(N)=O)c(=O)[nH]c3=O)cc2Cl)n[nH]c1=O]

Step 1. Concentrated aqueous HCl (10.0 mL, 1.0 v/w) was added to a stirred solution of 6-(4-amino-2,6-dichlorophenoxy)-4-isopropylpyridazin-3(2H)-one (compound 1) (10.0 g, 1.0 equiv.) in acetic acid (100 mL, 10.0 v/w). The resulting mixture was cooled to 5° C. and stirring for 15 min. NaNO₂ (2.4 g, 1.1 equiv.) was dissolved into H₂O (10 mL, 10.0 v/w), and this solution was added dropwise into the reaction mixture over 1 h at 5° C. The resulting mixture was stirred for at least 1 h at 5° C. Reaction completion was confirmed by HPLC. A solution of sodium acetate (6.6 g, 2.5 equiv.) and diethyl malonyldicarbamate (compound 2A-Et) (9.4 g, 1.2 equiv.) was added to the reaction mixture at 5° C. The reaction mixture was warmed to 25° C. and stirred for at least 1 h. Reaction completion was confirmed by HPLC. A solution of sodium acetate (2.1 g, 0.8 equiv.) was added to the reaction mixture at 25° C. The resulting mixture was heated to 80° C. and stirred for 2 h. Reaction completion was confirmed by HPLC. The mixture was cooled to 25° C. and stirred for 15 min. Concentrated aqueous HCl (50 mL, 10.0 v/w) was added to the reaction mixture at 25° C., which was then heated to 105° C. and stirred for 2 h. The reaction was confirmed to complete by HPLC. The resulting mixture was cooled to 25° C. and agitated for 1 h prior to filtration. The cake was washed with 1:1 AcOH/H₂O (20 mL, 2.0 v/w), prior to drying under nitrogen to afford 2-(3,5-dichloro-4-((5-isopropyl-6-oxo-1,6-dihydropyridazin-3-yl)oxy)phenyl)-3,5-dioxo-2,3,4,5-tetrahydro-1,2,4-triazine-6-carboxylic acid (compound 4) (8.7 g, 56.0% yield) with 93.1% purity. LCMS (ESI, m/z): 454 [M+H]⁺.

Step 2. Carbonyldiimidazole (2.6 g, 1.2 equiv.) was added to a stirred solution of compound 4 (6.0 g, 1.0 equiv.) in dimethylformamide (DMF) (90 mL, 15.0 v/w) and stirred for 30 min at 25° C. A solution of NH₃—H₂O (30 mL, 5.0 v/w) was added to the reaction mixture at 25° C., and the mixture was stirred for at least 1 h. Reaction completion was confirmed by HPLC. 4 M HCl was added to adjust pH to 7. After agitation for 1 h, the mixture was filtered. The resultant cake was washed with H₂O (12 mL, 2.0 v/w) and then dried under nitrogen to afford compound 5 (5.3 g, 87.8% yield) with 99.2% purity. LCMS (ESI, m/z): 453 [M+H]⁺.

### Example 6. Preparation of 2-(3,5-dichloro-4-((5-isopropyl-6-oxo-1,6-dihydropyridazin-3-yl)oxy)phenyl)-3,5-dioxo-2,3,4,5-tetrahydro-1,2,4-triazine-6-carboxamide (compound 5)—Option 5

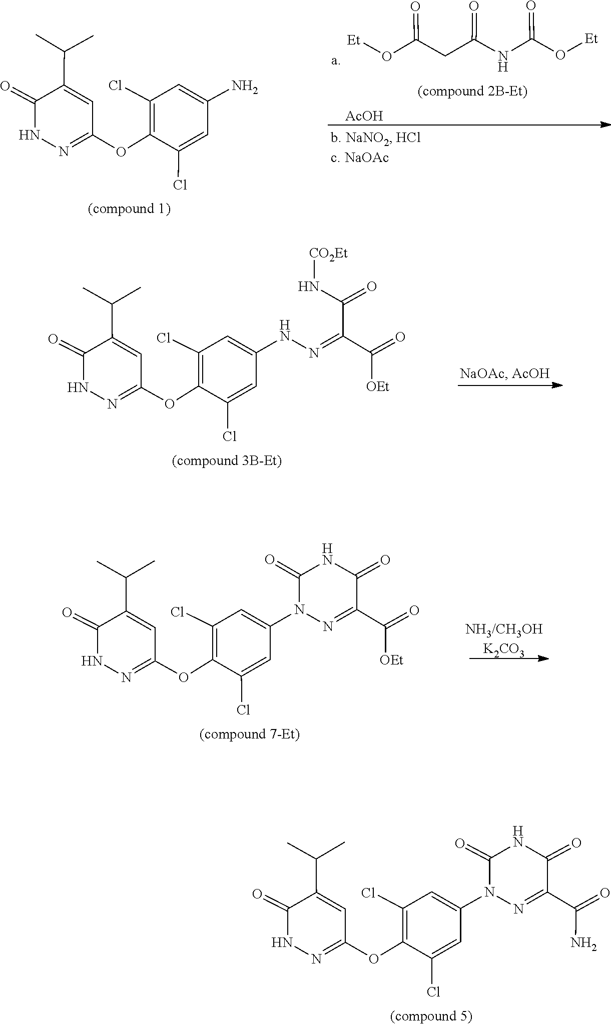 [SMILES: "AcOH" ; CC(C)c1cc(Oc2c(Cl)cc(N)cc2Cl)n[nH]c1=O ; CCOC(=O)CC(=O)NC(=O)OCC ; CCOC(=O)NC(=O)/C(=N\Nc1cc(Cl)c(Oc2cc(C(C)C)c(=O)[nH]n2)c(Cl)c1)C(=O)OCC ; CCOC(=O)c1nn(-c2cc(Cl)c(Oc3cc(C(C)C)c(=O)[nH]n3)c(Cl)c2)c(=O)[nH]c1=O ; CC(C)c1cc(Oc2c(Cl)cc(-n3nc(C(N)=O)c(=O)[nH]c3=O)cc2Cl)n[nH]c1=O]

Step 1. 6-(4-Amino-2,6-dichlorophenoxy)-4-isopropylpyridazin-3(2H)-one (compound 1) (1.00 g), ethyl 3-((ethoxycarbonyl)amino)-3-oxopropanoate (compound 2B-Et) (0.65 g), acetic acid (15 mL), and water (15 mL) were combined and stirred under an argon atmosphere. The mixture was cooled to 3° C. with an ice-water bath prior to addition of concentrated aqueous hydrogen chloride (0.8 mL). A solution of sodium nitrite (230 mg) in water (0.5 mL) was added dropwise while maintaining the temperature below 4° C. The resulting mixture was stirred for 30 minutes while kept cold. Sodium acetate trihydrate (1.30 g) was added to the reaction mixture, which was kept cold for another hour. The mixture was then warmed to room temperature and filtered. The filter cake was washed with 1:1 acetic acid:water (10 mL) and then with water (2×10 mL). The cake was dried at 65° C. to provide ethyl €-2-(2-(3,5-dichloro-4-((5-isopropyl-6-oxo-1,6-dihydropyridazin-3-yl)oxy)phenyl)hydrazineylidene)-3-((ethoxy-carbonyl)amino)-3-oxopropanoate (compound 3B-Et) (1.22 g). LCMS (ESI, m/z): 528 [M+H]⁺, eluting as 2 isomers.

Step 2. Compound 3B-Et (132 mg) was combined with sodium acetate (62 mg) and acetic acid (1.5 mL). The resultant mixture was heated to reflux under argon. After 2 hours and 15 minutes, the solution was cooled to room temperature, and water was added to the cloudpoint, initializing crystallization. The slurry was stirred for 1 hour and then filtered. The filter cake was washed with water and then dried at 70° C. under vacuum to afford ethyl 2-(3,5-dichloro-4-((5-isopropyl-6-oxo-1,6-dihydropyridazin-3-yl)oxy)phenyl)-3,5-dioxo-2,3,4,5-tetrahydro-1,2,4-triazine-6-carboxylate (compound 7-Et) (100 mg). ¹H NMR (d₆-DMSO): 12.77 (s, 1H), 12.20 (s, 1H), 7.80 (s, 2H), 7.45 (s, 1H), 4.33 (q, 2H), 3.08 (m, 1H), 1.25 (t, 3H), 1.20 (d, 6H). LCMS (ESI, m/z): 482 [M+H]⁺.

Step 3. Compound 7-Et (94 mg) was combined with 7 N NH₃ in methanol (2 mL), stirred for 55 hours and then concentrated. Acetic acid (1 mL) was added, and then the solution was concentrated. Methanol (2 mL) was added, followed by triethylamine (73 mg). A solution of acetic acid (59 mg) in methanol (0.5 mL) was added dropwise with stirring, resulting in a crystallization that began after a few minutes. After 30 minutes, the slurry was filtered, and the filter cake washed with methanol and dried at 70° C. under vacuum. Analytical data matched that provided above for compound 5.

### Example 7. Preparation of 2-(3,5-dichloro-4-((5-isopropyl-6-oxo-1,6-dihydropyridazin-3-yl)oxy)phenyl)-3,5-dioxo-2,3,4,5-tetrahydro-1,2,4-triazine-6-carboxamide (compound 5)—Option 6

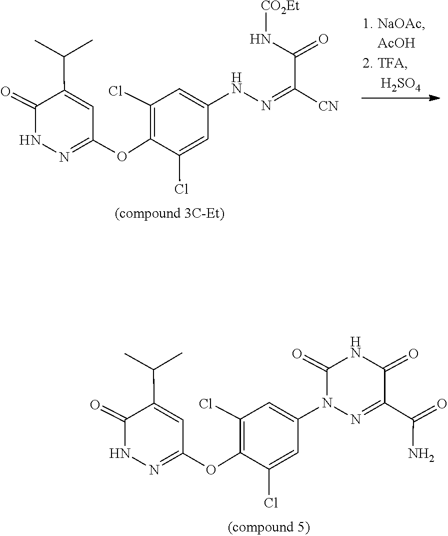 [SMILES: CCOC(=O)NC(=O)/C(C#N)=N\Nc1cc(Cl)c(Oc2cc(C(C)C)c(=O)[nH]n2)c(Cl)c1 ; CC(C)c1cc(Oc2c(Cl)cc(-n3nc(C(N)=O)c(=O)[nH]c3=O)cc2Cl)n[nH]c1=O]

Ethyl (Z)-(2-cyano-2-(2-(3,5-dichloro-4-((5-isopropyl-6-oxo-1,6-dihydro-pyridazin-3-yl)oxy)phenyl)hydrazineylidene)acetyl)carbamate (compound 3C-Et) was prepared as described in WO 2007/009913.

Compound 3C-Et (18.00 g, 1.0 equiv.) was combined under argon with sodium acetate (9.20 g, 3.0 equiv.) and acetic acid (90 mL), stirred, and heated to an internal temperature (Tᵢₙₜ) of 117° C. After 25 minutes, the slurry had transformed to a homogenous solution. After 70 minutes, a sample was withdrawn, diluted with acetonitrile and DMSO, and analyzed by HPLC-MS: all starting material had converted to either a cyclized nitrile-containing intermediate or compound 5. The reaction was cooled to Tᵢₙₜ '₂ 30° C. using a cool-water bath prior to addition of trifluoroacetic acid (180 mL) so that Tᵢₙₜ<33° C. While cooling below 32° C., 99.999% sulfuric acid (90 mL) was added. The purple, homogeneous solution was heated to Tᵢₙₜ '₂ 80° C. During the ensuing reaction to compound 5, HPLC-MS analyses were taken by withdrawing samples and diluting them with acetonitrile and then DMSO. After 2 hours, the relative integrated absorbances [254 nm] of the cyclized nitrile-containing intermediate, compound 5, and the carboxylic acid produced by further hydrolysis of the amide functionality were, respectively, 1.8, 97.7, and 0.5. Using ice-water cooling so that Tᵢₙₜ<33° C., water (360 mL) was added dropwise followed by seeds of compound 5. After stirring at room temperature for 50 minutes, ice-water cooling was applied for 30 minutes. The resultant slurry was filtered, washed with 4:1 water:acetic acid (70 mL), then with water (50 mL). The filter cake was dried under vacuum at 65° C. to provide crude compound 5 (12.10 g). This crude material was mixed with methanol (360 mL) and triethylamine (8.10 g, 3.0 equiv.). The mixture was stirred for 20 minutes, dissolving all solids. Acetic acid (6.41 g, 4.0 equiv.) was added over 1 minute. After 1 minute of stirring, recrystallization of compound 5 initiated. The slurry was stirred for 1 hour, filtered, and washed with methanol (50 mL). The filter cake was dried under vacuum at 65° C. to provide compound 5 (11.41 g, 67% yield over 2 reactions). Purity=98.6%, with 0.11% of the cyclized nitrile and 0.42% of the carboxylic acid impurities described earlier.

### Example 8. Preparation of Compound a (Kilogram-Scale Batches)

The preparation of compound A from 2-(3,5-dichloro-4-((5-isopropyl-6-oxo-1,6-dihydropyridazin-3-yl)oxy)phenyl)-3,5-dioxo-2,3,4,5-tetrahydro-1,2,4-triazine-6-carboxamide (compound 5) was conducted on a kilogram scale, starting from 2.6 kg, 1.8 kg, 2.6 kg, 1.8 kg, and 1.7 kg of compound 5.

A solution of sodium hydroxide (3.5 equiv.) in water (17 v/w) was prepared in a 50-L or 80-L reactor at room temperature (25±5° C.) under a nitrogen atmosphere and cooled to 0-5° C. prior to the addition of 2-(3,5-dichloro-4-((5-isopropyl-6-oxo-1,6-dihydropyridazin-3-yl)oxy)phenyl)-3,5-dioxo-2,3,4,5-tetrahydro-1,2,4-triazine-6-carboxamide (compound 5) (1 equiv.). A bleach solution (sodium hypochlorite, 8-10% available chlorine) (2.4 equiv.) was added dropwise to the reactor over a one-hour period. The resultant mixture was stirred at 0±5° C. for 90 min and then heated to 35-45° C. for 6 hours. The reaction was judged complete by HPLC analysis. After cooling the mixture to 0-10° C., the pH was adjusted to 5-6 using 2 M HCl. Crude product was collected by filtration from each of the five batches.

The filter cakes from the five batches were combined and suspended as a slurry in THF/acetic acid (10:1, v/v) and filtered. The resultant filter cake was then suspended in acetic acid and filtered, and this process was repeated five times to afford compound A (5.21 kg, 99.6% purity).

### REFERENCES

1. Younossi, Z M, Koenig, A B, Abdelatif, D, Fazel, Y, Henry, L, Wymer, M. Global epidemiology of nonalcoholic fatty liver disease-Meta-analytic assessment of prevalence, incidence, and outcomes. Hepatology, 2016, 64(1):73e84.
2. Gastroenterology. 2012 June; 142(7): 1592-609. Doi: 10.1053/j.gastro.2012.04.001. Epub 2012 May 15.
3. Serfaty, L., Lemoine, M. Definition and natural history of metabolic steatosis: clinical aspects of NAFLD, NASH and cirrhosis. Diabetes and Metabolism, 2008, 34 (6 Pt 2):634e637.
4. Hepatology. 2012 October; 56(4): 1580-1584. Doi: 10.1002/hep.26031
5. Dulai, P S, Singh, S, Patel, J, Soni, M, Prokop, L J, Younossi, Z, et al., Increased risk of mortality by fibrosis stage in nonalcoholic fatty liver disease: systematic review and meta-analysis. Hepatology, 2017, 65(5):1557e1565.
6. Younossi, Z M, Loomba, R, Rinella, M E, Bugianesi, E, Marchesini, G, Neuschwander-Tetri, B A, et al. Current and future therapeutic regimens for non-alcoholic fatty liver disease (NAFLD) and non-alcoholic steatohepatitis (NASH). Hepatology, 2018, 68(1):349e360.
7. Harvey C B, Williams G R. Mechanism of thyroid hormone action. Thyroid, 2002 June; 12(6):441-6.
8. Bookout A L, Jeong Y, Downes M, Yu R T, Evans R M, Mangelsdorf D J. Anatomical profiling of nuclear receptor expression reveals a hierarchical transcriptional network. Cell, 2006, 126:789-799
9. Flamant F, Baxter J D, Forrest D, Refetoff S, Samuels H H, Scanlan T S, Vennstrom B, Samarut J. International union of pharmacology. LIX. The pharmacology and classification of the nuclear receptor superfamily: thyroid hormone receptors. Pharmacol. Rev., 2006, 58:705-711
10. Haning H, Woltering M, Mueller U, Schmidt G, Schmeck C, Voehringer V, Kretschmer A, Pernerstorfer J. Bioorg. Med Chem Lett., 2005 Apr. 1, 15(7): 1835-40. Novel heterocyclic thyromimetics.
11. Hirano T, Kagechika H. Thyromimetics: a review of recent reports and patents (2004-2009). Expert Opin Ther Pat., 2010 February; 20(2):213-28. Doi: 10.1517/13543770903567069.
12. Kowalik M A, Columbano A, Perra A. Thyroid Hormones, Thyromimetics and Their Metabolites in the Treatment of Liver Disease. Front Endocrinol (Lausanne), 2018 Jul. 10; 9:382. Doi: 10.3389/fendo.2018.00382. eCollection 2018.
13. Hartley M D, Kirkemo L L, Banerji T, Scanlan T S. A Thyroid Hormone-Based Strategy for Correcting the Biochemical Abnormality in X-Linked Adrenoleukodystrophy. Endocrinology 2017, 158(5), p 1328-1338. Doi: 10.1210/en.2016-1842.
14. Milanesi A, Brent G A. Beam Me In: Thyroid Hormone Analog Targets Alternative Transporter in Mouse Model of X-Linked Adrenoleukodystrophy. Endocrinology 2017, 158, p 1116-1119. Doi: 10.1210/en.2017-00206.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

1. A method to prepare compound A: 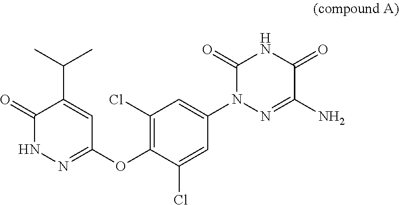 [SMILES: CC(C)c1cc(Oc2c(Cl)cc(-n3nc(N)c(=O)[nH]c3=O)cc2Cl)n[nH]c1=O]
wherein the method comprises converting compound 5: 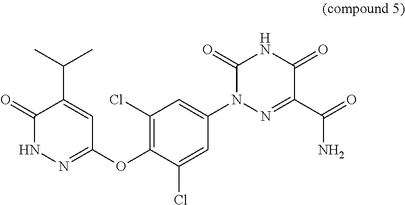 [SMILES: CC(C)c1cc(Oc2c(Cl)cc(-n3nc(C(N)=O)c(=O)[nH]c3=O)cc2Cl)n[nH]c1=O]
to compound A.

2. The method of claim 1, wherein compound 5 is converted to compound A in a one-pot reaction.

3. The method of claim 2, wherein compound 5 is converted to compound A in a single-step reaction.

4. The method of claim 1, wherein compound 5 is converted to compound A under reaction conditions comprising contacting compound 5 with an oxidant and a base.

5. The method of claim 4, wherein the oxidant comprises NaOCl.

6. The method of claim 4, wherein the base comprises NaOH.

7. The method of claim 1, wherein compound 5 is converted to compound A under inert gas.

8. The method of claim 1, further comprising reacting compound 1: 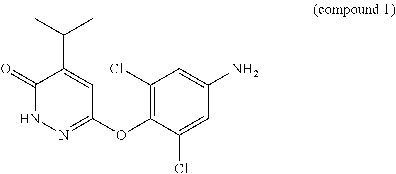 [SMILES: CC(C)c1cc(Oc2c(Cl)cc(N)cc2Cl)n[nH]c1=O]
with compound 2A: 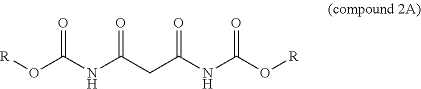
wherein R is C₁-C₃ alkyl or benzyl, under diazotization conditions to prepare compound 3A:

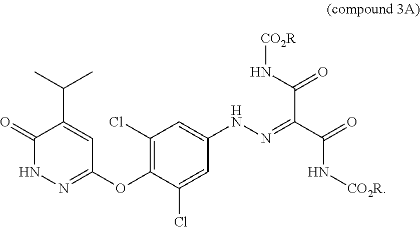

9. The method of claim 8, wherein the diazotization conditions comprise nitrite salt and one or more acids.

10. The method of claim 8, wherein the diazotization conditions are under inert gas.

11. The method of claim 8, further comprising cyclizing compound 3A to prepare compound 5 in a one-pot step comprising heating compound 3A under basic conditions.

12. The method of claim 11, wherein the basic conditions are under inert gas.

13. The method of claim 11, wherein the basic conditions comprise ammonia.

14. The method of claim 13, wherein the basic conditions further comprise potassium carbonate.

15. The method of claim 11, wherein the one-pot step comprises heating compound 3A at about 45° C. with ammonia and potassium carbonate in methanol.

16. The method of claim 8, further comprising cyclizing compound 3A to prepare compound 6:

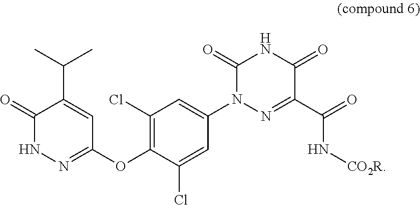

17.-27. (canceled)

28. The method of claim 1, further comprising reacting compound 1: 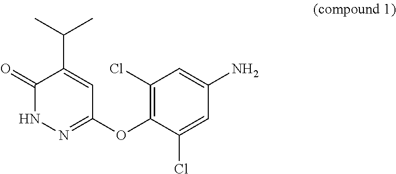 [SMILES: CC(C)c1cc(Oc2c(Cl)cc(N)cc2Cl)n[nH]c1=O]
with compound 2A: 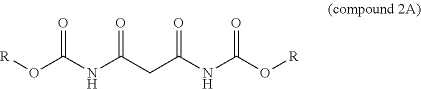
wherein R is C₁-C₃ alkyl or benzyl, under diazotization conditions followed by cyclizing under acidic conditions in a one-pot reaction to prepare compound 4:

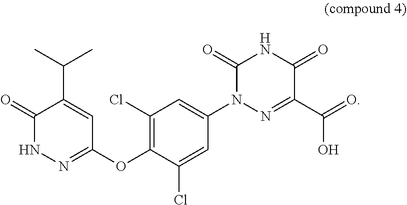 [SMILES: CC(C)c1cc(Oc2c(Cl)cc(-n3nc(C(=O)O)c(=O)[nH]c3=O)cc2Cl)n[nH]c1=O]

29.-34. (canceled)

35. The method of claim 1, further comprising reacting compound 1: 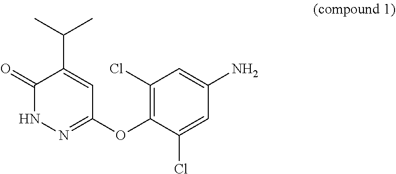 [SMILES: CC(C)c1cc(Oc2c(Cl)cc(N)cc2Cl)n[nH]c1=O]
with compound 2B: 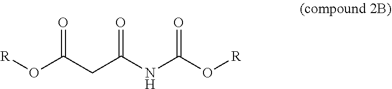
wherein R is C₁-C₃ alkyl or benzyl, under diazotization conditions to prepare compound 3B:

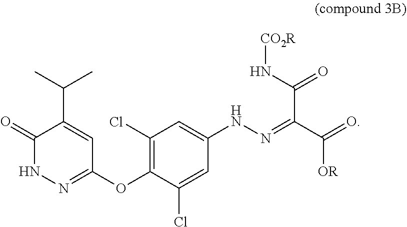

36.-37. (canceled)

38. The method of claim 35, further comprising cyclizing compound 3B to prepare compound 7:

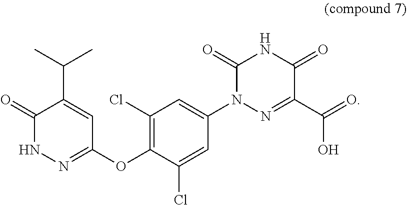 [SMILES: CC(C)c1cc(Oc2c(Cl)cc(-n3nc(C(=O)O)c(=O)[nH]c3=O)cc2Cl)n[nH]c1=O]

39.-47. (canceled)

48. The method of claim 1, further comprising cyclizing compound 3C: 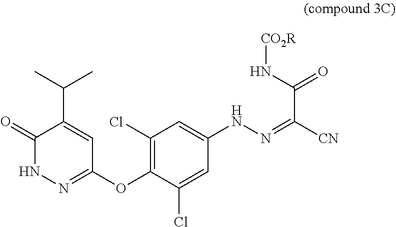
to prepare compound 5 in a one-pot step.

49.-80. (canceled)

81. A method to prepare compound A: 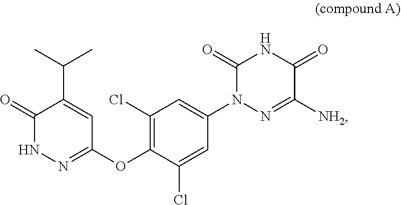 [SMILES: CC(C)c1cc(Oc2c(Cl)cc(-n3nc(N)c(=O)[nH]c3=O)cc2Cl)n[nH]c1=O]
wherein the method comprises:
(1) adding bleach to a cooled aqueous solution of sodium hydroxide and compound 5: 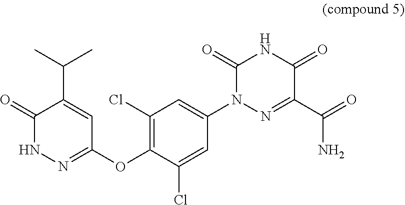 [SMILES: CC(C)c1cc(Oc2c(Cl)cc(-n3nc(C(N)=O)c(=O)[nH]c3=O)cc2Cl)n[nH]c1=O]
to form a reaction mixture;
(2) stirring the reaction mixture while cooled with an ice bath;
(3) stirring the reaction mixture at 35-45° C.;
(4) adjusting pH of the reaction mixture to pH of 5-6, with stirring, to form a slurry; and
(5) isolating compound A from the slurry by filtration.

82. The method of claim 81, wherein:
(i) the cooled aqueous solution of sodium hydroxide and compound 5 is cooled to 4° C. to 5° C.;
(ii) the reaction mixture is formed under inert gas;
(iii) the reaction mixture is stirred for about 90 minutes while cooled with an ice bath;
(iv) the reaction mixture is stirred for about 6 hours at 35-45° C.; and
(v) pH of the reaction mixture is adjusted to 5-6 using hydrochloric acid.

83.-86. (canceled)

87. The method of claim 81, wherein the method further comprises triturating compound A using acetic acid and a combination of THE and acetic acid.

88. A method to prepare compound A: 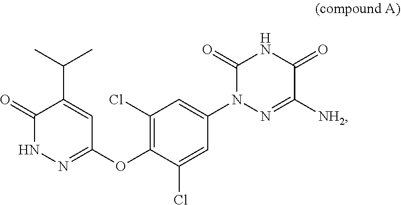 [SMILES: CC(C)c1cc(Oc2c(Cl)cc(-n3nc(N)c(=O)[nH]c3=O)cc2Cl)n[nH]c1=O]
wherein the method comprises:
(1) adding acetic acid, water, and aqueous HCl to compound 1 and compound 2A-Et: 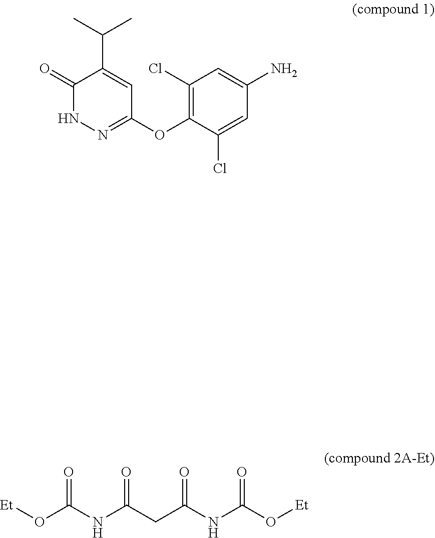 [SMILES: CC(C)c1cc(Oc2c(Cl)cc(N)cc2Cl)n[nH]c1=O ; CCOC(=O)NC(=O)CC(=O)NC(=O)OCC]
to form an initial reaction mixture;
(2) adding an aqueous solution of sodium nitrite to the initial reaction mixture to form a second reaction mixture while maintaining the temperature at room temperature;
(3) stirring the second reaction mixture at room temperature;
(4) adding sodium acetate to the second reaction mixture while maintaining the temperature at no more than 30° C. to form a third reaction mixture;
(5) stirring the third reaction mixture at room temperature;
(6) isolating compound 3A-Et: 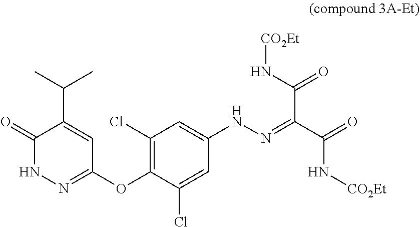 [SMILES: CCOC(=O)NC(=O)C(=NNc1cc(Cl)c(Oc2cc(C(C)C)c(=O)[nH]n2)c(Cl)c1)C(=O)NC(=O)OCC]
by filtration;
(7) adding ammonia in methanol to compound 3A-Et and potassium carbonate to form a fourth reaction mixture;
(8) stirring the fourth reaction mixture at about 45° C.;
(9) adjusting the pH of the fourth reaction mixture to pH of 5-6 while maintaining the temperature at room temperature;
(10) isolating compound 5: 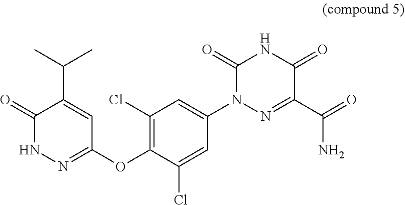 [SMILES: CC(C)c1cc(Oc2c(Cl)cc(-n3nc(C(N)=O)c(=O)[nH]c3=O)cc2Cl)n[nH]c1=O]
by filtration;
(11) adding bleach to a cooled aqueous solution of sodium hydroxide and compound 5 to form a fifth reaction mixture;
(12) stirring the fifth reaction mixture while cooled with an ice bath;
(13) stirring the fifth reaction mixture at 35-45° C.;
(14) adjusting pH of the fifth reaction mixture to pH of 5-6, with stirring, to form a slurry; and
(15) isolating compound A from the slurry by filtration.

89. The method of claim 88, wherein:
(i) the initial reaction mixture is formed under inert gas,
(ii) the second reaction mixture is stirred for about 30 minutes at room temperature:
(iii) the third reaction mixture is stirred overnight at room temperature;
(iv) the fourth reaction mixture is formed under inert gas; and
(v) the fourth reaction mixture is stirred for at least 18 hours at about 45° C.

90.-93. (canceled)

94. The method of claim 88, wherein the method further comprises triturating compound 5, optionally wherein compound 5 is triturated using THF.

95. (canceled)

96. The method of claim 88, wherein:
(i) the cooled aqueous solution of sodium hydroxide and compound 5 is cooled to 4° C. to 5° C.;
(ii) the fifth reaction mixture is formed under inert gas;
(iii) the fifth reaction mixture is stirred for about 90 minutes while cooled with an ice bath;
(iv) the fifth reaction mixture is stirred for about 6 hours at 35-45° C.; and
(v) the pH of the fifth reaction mixture is adjusted to pH of 5-6 using hydrochloric acid.

97.-100. (canceled)

101. The method of claim 88, wherein the method further comprises triturating compound A using acetic acid and a combination of THE and acetic acid.

102. A compound having the structure of: 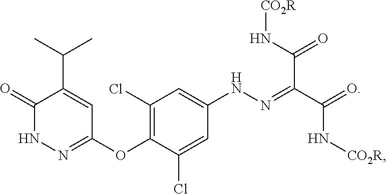
wherein R is C₁-C₃ alkyl or benzyl.

103-104. (canceled)
